(19) United States
(12) Patent Application Publication
(10) Pub. No.: US 2024/0277285 A1
(43) Pub. Date: Aug. 22, 2024

(54) SYSTEMS AND METHODS FOR DIAGNOSIS AND/OR TREATING DEMYELINATING NEUROPATHY

(71) Applicant: Rutgers, The State University of New Jersey, New Brunswick, NJ (US)

(72) Inventor: Nizar Souayah, New Brunswick, NJ (US)

(21) Appl. No.: 18/651,381

(22) Filed: Apr. 30, 2024

Related U.S. Application Data
(63) Continuation of application No. PCT/US2022/049119, filed on Nov. 7, 2022.
(60) Provisional application No. 63/276,989, filed on Nov. 8, 2021.

(51) Int. Cl. A61B 5/00; A61B 5/145; A61B 5/262; A61B 5/296; A61B 5/397; G16H 20/10; G16H 80/00

(52) U.S. Cl. CPC A61B 5/4836; A61B 5/14546; A61B 5/262; A61B 5/296; A61B 5/397; A61B 5/7267; A61B 5/742; G16H 20/10; G16H 80/00

(57) ABSTRACT

In some embodiments, the present disclosure provides examples of a method for treating a demyelinating neuropathy. Electrodes can be placed on a muscle area of a patient to acquire a compound muscle action potential (CMAP) measure of the muscle integrity of the patient. A biological sample of the patient can be obtained. The patient can be identified as having a demyelinating neuropathy by inputting a conduction measure associated with a CMAP amplitude comprised in the CMAP measure into a regression model to determine a conduction measure that is within a demyelinating range defined by the regression model and determining, from the biological sample of the patient, a biomarker activity measure of the patient that is above a predetermined threshold. The method includes administering to the patient identified as having a demyelinating neuropathy a therapeutically effective amount of at least one agent used to treat the demyelinating neuropathy.

### CROSS-REFERENCE TO RELATED APPLICATIONS

This application is a Continuation Application relating to and claiming the benefit of commonly-owned, co-pending PCT International Application No. PCT/US2022/049119, filed Nov. 7, 2022, which claims priority to and the benefit of to U.S. Provisional Application No. 63/276,989 filed Nov. 8, 2021 and titled “METHODS FOR DIAGNOSIS AND TREATING DEMYELINATING NEUROPATHY,” the contents of each of the foregoing are herein incorporated by reference in their entirety.

### FIELD OF TECHNOLOGY

The present disclosure generally relates to systems and methods for diagnosis and/or treating demyelinating neuropathy in a patient using regression models and neuroinflammatory biomarkers.

### BACKGROUND OF TECHNOLOGY

Demyelinating neuropathies are varied in their clinical characteristics. In etiology they may be inherited or acquired, in their time course, acute or chronic, and in their distribution, multifocal or generalized. Demyelinating neuropathies can be characterized by depressed reflexes, weakness, discriminative sensory loss, and slowed nerve conduction velocities.

Demyelination may occur in distal symmetrical polyneuropathy (DSP) which is the most common form of diabetic neuropathy, one of the most frequent chronic complications of diabetes mellitus and one of the major causes of limb amputation.

Some pathological studies have concluded that diabetic DSP is mainly an irreversible neuropathy with axonal degeneration, regeneration, and to a lesser degree, secondary demyelination. Slowness of conduction velocity (CV) in DSP exceeds that observed in distal axonal non-diabetic neuropathy. In addition, axonal atrophy, a characteristic of chronic axonal loss, does not occur to any degree in diabetic DSP. Motor nerve conduction slowing in diabetic DSP can be attributed to an additional demyelination rather than a pure loss of the fastest conducting large, myelinated fibers.

### SUMMARY OF DESCRIBED SUBJECT MATTER

In some embodiments, the present disclosure provides examples of a method for treating demyelinating neuropathy of a patient. Electrodes can be placed on a muscle area of the patient to acquire a compound muscle action potential (CMAP) after stimulation of corresponding peripheral nerve to measure the muscle integrity. A biological sample of the patient can be obtained, for example, urine. The patient can be identified as having a demyelinating neuropathy by inputting a conduction measure associated with a CMAP amplitude comprised in the CMAP measure into a regression model to determine a conduction measure that is within a demyelinating range defined by the regression model and determining, from the biological sample of the patient, a biomarker activity measure of the patient that is above a predetermined threshold. The method includes administering to the patient identified as having a demyelinating neuropathy a therapeutically effective amount of at least one agent used to treat the demyelinating neuropathy.

In some embodiments, the patient can be identified as having a distal symmetric polyneuropathy, a peripheral neuropathy, or a chronic inflammatory demyelinating polyneuropathy.

In some embodiments, the biomarker can be a neuroinflammatory biomarker, for example, an enzyme and more specifically a secretory phospholipase 2 (sPLA2) enzyme.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIGS. 1-11 show one or more plots, and schematic flow diagrams, which are illustrative of some aspects of at least some embodiments of the present disclosure.

FIG. 1 illustrates a method for diagnosis and treatment of demyelinating neuropathy, in accordance with embodiments of the present disclosure.

FIG. 2A, FIG. 2B, FIG. 2C and FIG. 2D illustrate converted data for CMAP amplitude versus conduction velocity for the tibial, peroneal, median, and ulnar nerve from a group of 95 subjects diagnosed with amyotrophic lateral sclerosis (ALS).

FIG. 3A, FIG. 3B, FIG. 3C and FIG. 3D illustrate converted data for CMAP amplitude versus distal latency for the tibial, peroneal, median, and ulnar nerve from a group of 95 subjects diagnosed with amyotrophic lateral sclerosis (ALS).

FIG. 4A, FIG. 4B, FIG. 4C and FIG. 4D illustrate converted data for CMAP amplitude versus F responses for the tibial, peroneal, median, and ulnar nerve from a group of 95 subjects diagnosed with amyotrophic lateral sclerosis (ALS).

FIG. 5A, FIG. 5B, FIG. 5C and FIG. 5D illustrate regression plots of converted normalized CV (y-axis, dependent variable) and converted normalized CMAP amplitude (x-axis, independent variable) for the diabetic DSP, non-diabetic DSP, and ALS groups.

FIG. 6A, FIG. 6B, FIG. 6C and FIG. 6D illustrate regression plots of transformed CMAP amplitudes (x-axis, independent variable) for the A, B, and ALS groups.

FIG. 7 is a block diagram of an exemplary computer-based system for demyelinating neuropathy detection and/or diagnosis in accordance with one or more embodiments of the present disclosure.

FIG. 8 depicts a block diagram of an exemplary computer-based system and platform 800 in accordance with one or more embodiments of the present disclosure.

FIG. 9 depicts a block diagram of another exemplary computer-based system and platform 900 in accordance with one or more embodiments of the present disclosure.

FIG. 10 illustrates schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

FIG. 11 illustrates schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Typically, nerve conduction studies cannot definitively distinguish between irreversible large-fiber loss and potentially reversible, neuroinflammation-associated demyelination. Both human studies and animal models have implicated a complex of interdependent metabolic, neurochemical, inflammatory, and vascular processes involved in diabetic DSP pathogenesis.

Acquired demyelination may occur affect any segment of the peripheral nerve causing peripheral neuropathy. Peripheral neuropathy is a condition that refers to damage to the peripheral nervous system with nerve signaling disruption that can include loss of signal, inappropriate signaling, and distortion in signaling. Symptoms can range from mild to disabling, and can develop over days, weeks, or years. Peripheral neuropathy can affect one nerve (mononeuropathy), two or more nerves (multiple mononeuropathy or mononeuropathy multiplex), with many or most nerves affected called polyneuropathy. The condition is often misdiagnosed given the complexity of symptoms. Not all patients with symptoms of neuropathy are tested for peripheral neuropathy and testing may not assess for all forms of neuropathy. Peripheral neuropathies have varying symptoms dependent on the type of nerve involved and damage; motor, sensory, or autonomic. Most peripheral neuropathies affect all three types of nerve fibers, with others affecting one or two. Three-fourths of polyneuropathies are length-dependent with the farthest nerve endings in the feet where symptoms develop first, with the more severe presentations moving upwards to the central part of the body. In non-length dependent polyneuropathies symptoms move towards torso or are patchy.

Chronic inflammatory demyelinating polyneuropathy is another example of demyelinating neuropathy. Chronic inflammatory demyelinating polyneuropathy (CIDP) is a neurological disorder in which there is inflammation of nerve roots and peripheral nerves and destruction of the fatty protective covering (myelin sheath) over the nerves. This affects how fast the nerve signals are transmitted and leads to loss of nerve fibers. This causes weakness, paralysis and/or impairment in motor function, especially of the arms and legs (limbs). Sensory disturbance may also be present. The motor and sensory impairments usually affect both sides of the body (symmetrical), and the degree of severity and the course of disease may vary from case to case. Some affected individuals may follow a slow steady pattern of symptoms while others may have symptoms that stabilize and then relapse.

In some embodiments, diagnosing and treat a demyelinating neuropathy may be improved by leveraging enhanced expression of inflammatory biomarkers that may occur in subjects having demyelinating neuropathy. One example of such biomarkers is the phospholipase A2 enzyme family. Secretory phospholipase A2 (sPLA2) expression is increased in type 2 diabetic patients related to metabolic syndrome. The level of sPLA2 is higher in diabetic patients with macroangiopathy compared to those without macroangiopathy. Consequently, sPLA2 is a potential neuroinflammatory biomarker for diabetic DSP.

In some embodiments, clinical examination and conventional nerve conduction studies may be insufficient to make a clear distinction between loss of large heavily myelinated fibers and minor degrees of demyelination. Thus, in clinical examination and/or conventional nerve conduction studies, the motor nerve conduction slowing from primary demyelination in diabetic neuropathy is unlikely to be recognized as such, until the motor nerve conduction slowing reaches a severity such that it can be clearly attributed to a process other than pure axonal loss with secondary myelinopathy.

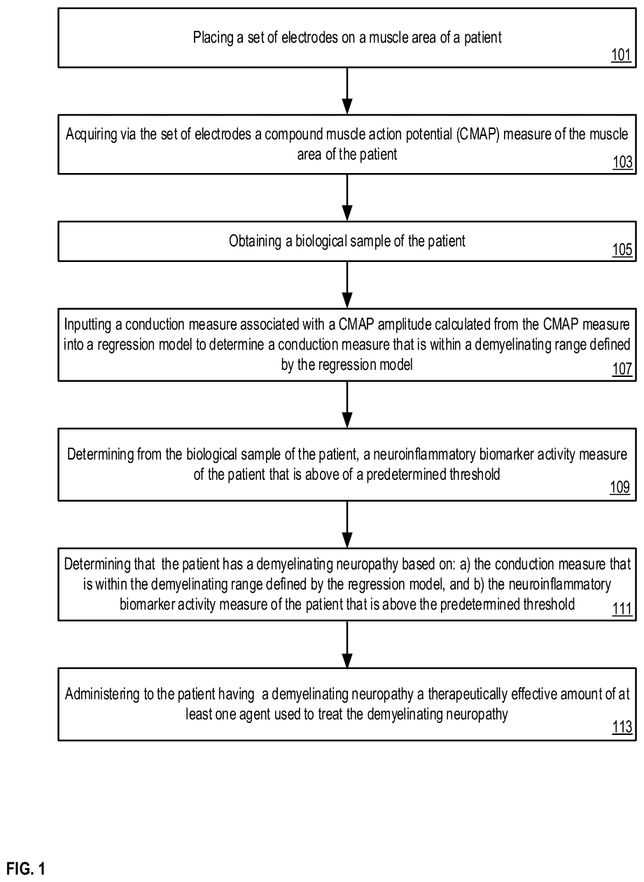

Accordingly, in some embodiments, improved techniques for identifying diabetic patients with DSP neuropathy may enable more accurate diagnosis and treatment at an early stage. FIGS. 1 through 11 illustrate systems and methods of electromyography for improved diagnosis and treatment of demyelinating neuropathy. The following embodiments provide technical solutions and/or technical improvements that overcome technical problems, drawbacks and/or deficiencies in the technical fields inability to assess for all types of neuropathy and insufficient techniques for early and accurate detection of demyelination as detailed above. As explained in more detail, below, technical solutions and/or technical improvements herein include aspects of improved demyelination detection via compound muscle action potential (CMAP) using electromyography for more sensitive and accurate detection of demyelination, thus enabling improved diagnosis and treatment of demyelinating neuropathy. Based on such technical features, further technical benefits become available to users and operators of these systems and methods. Moreover, various practical applications of the disclosed technology are also described, which provide further practical benefits to users and operators that are also new and useful improvements in the art.

FIG. 1 illustrates a method for diagnosis and treatment of demyelinating neuropathy, in accordance with embodiments of the present disclosure. Demyelinating neuropathies can be acute, subacute, or chronic. Demyelinating neuropathies are characterized by depressed reflexes, weakness, discriminative sensory loss, and slowed nerve conduction velocities.

In some embodiment, electrodes are used to gather electrodiagnostic signals from the patient, for example, electromyography. Electromyography is a technique for evaluating and recording the electrical activity produced by skeletal muscles. A clinician or other healthcare provider can place a set of electrodes on a muscle area of a patient as shown at 101. In some embodiments, the electrodes can be surface electrodes, for example, a single surface electrode, a pair of surface electrodes or an array of multiple surface electrodes. In some embodiments, the electrodes can be needle-based electrodes. In some embodiments, needle-based electrodes can be used to perform intramuscular electromyography. Similar to the surface electrodes, different number of needle-based electrodes can be placed on the patient, for example, one needle-based electrode, a pair of needle-based electrodes, or an array of multiple needle-based electrodes. Some examples of needle-based electrodes include monopolar needle electrodes, concentric needle electrodes, single-fiber needle electrode, and other suitable needle-based electrodes.

In some embodiments, the muscle area of the patient can be any muscle area that can be used to capture electrical activity produced by skeletal muscles for example, the tibial nerve, the peroneal nerve, the median nerve, the ulnar nerve, the radial nerve, the sural nerve, or other suitable muscle areas.

In some embodiments, a compound muscle action potential (CMAP) can be acquired via the set of electrodes placed on the muscle area of the patient as shown at 103. The CMAP measure indicates a summation of a group of almost simultaneous action potentials from several muscle fibers located in the same muscle area of the patient. In some embodiments, latency, amplitude, duration, and area of the CMAP measure can be calculated. An action potential occurs when the membrane potential of a specific cell location rapidly rises and falls: this depolarization then causes adjacent locations to similarly depolarize. Action potentials occur in certain patient cells, called excitable cells, for example, neurons, muscle cells, endocrine cells, glomus cells, and other excitable cells.

In some embodiments, a biological sample can be obtained from the patient as shown at 105. For example, a urine sample can be obtained from the patient to measure biomarkers activity contained in the urine sample. Examples of such biomarkers are secretory phospholipase 2 (sPLA2) enzymes or other suitable enzyme which control processes in skin and other organs, including inflammation and differentiation.

In some embodiments, a conduction measure, for example, conduction velocity associated with a CMAP amplitude can be calculated from the CMAP measure. In some embodiments, a conduction measure such as the conduction velocity can be calculated as the difference of a proximal latency and distal latency and dividing the result by the distance between the proximal latency stimulating point and the distal latency stimulating point. In some embodiments, the conduction measure (e.g., conduction velocity or other suitable conduction measure as described above) can be inputted into a regression model to determine a conduction measure that is within a demyelinating range that is defined by the regression model as shown at 107. Table 1 below provides examples of AAN criteria for regression models to provide a range of values for demyelination for CIDP, DSP and peripheral neuropathy. In some embodiments, columns for Min DL, Max CV and Min F correspond to the regression model being used.

TABLE 1
Conduction slowing using the regression equations and AAN criteria.
For each studied nerve, a CMAP amplitude equal to 50% and 100%
of lower limit of normal, and a height of 65 inches was used.

| | | | | | |
|---|---|---|---|---|---|
| | Min DL | | Max CV | | Min F |
| Min | by AAN | Max | by AAN | Min | by AAN |
| DL | criterion | CV | criterion | F | criterion |
| (ms) | (ms) | (m/s) | (m/s) | (ms) | (ms) |

CMAP 100%; F for height 65″

| | | | | | | |
|---|---|---|---|---|---|---|
| Median nerve | 4.7 | 5.6 | 41.9 | 39.2 | 33.8 | 34.8 |
| Ulnar nerve | 3.7 | 5.0 | 48.8 | 37.6 | 33.2 | 36.2 |
| Tibial nerve | 7.3 | 8.8 | 34.3 | 29.6 | 62.2 | 68.4 |
| Peroneal nerve | 6.7 | 8.1 | 37.2 | 31.2 | 59.8 | 68.4 |

CMAP 50%; F for height 65″

| | | | | | | |
|---|---|---|---|---|---|---|
| Median nerve | 5.4 | 6.8 | 39.0 | 34.3 | 35.9 | 43.5 |
| Ulnar nerve | 4.3 | 6.0 | 41.7 | 32.9 | 37.7 | 45.3 |
| Tibial nerve | 7.3 | 10.5 | 33.2 | 25.9 | 63.4 | 85.5 |
| Peroneal nerve | 6.8 | 9.8 | 36.6 | 27.3 | 60.2 | 85.5 |

CMAP, compound muscle action potential; CV, conduction velocity; DL, distal latency; F; F latency.
Min DL: minimum distal latency value per the regression equation, above which it considered in the demyelination range.
Min DL by AAN criterion: minimum distal latency value, above which it considered in the demyelination range by AAN criteria.
Max CV by AAN criteria: Upper limit of conduction velocity below which the conduction velocity is considered in the demyelination.

In some embodiments, the regression model ingests a feature vector that encodes features representative of CMAP measurements (e.g., CMAP amplitude, conduction velocity, or other CMAP measurement or any combination thereof). In some embodiments, the regression model processes the feature vector with parameters to produces a prediction of a demyelination value range indicative of a degree of demyelination for CIDP, DSP and peripheral neuropathy, e.g., as measured by conduction slowing. In some embodiments, the parameters of the regression model may be implemented in a suitable machine learning model including a regression machine learning model, such as, e.g., Linear Regression, Logistic Regression, Ridge Regression, Lasso Regression, Polynomial Regression, Bayesian Linear Regression (e.g., Naive Bayes regression), a recurrent neural network (RNN), decision trees, random forest, support vector machine (SVM), or any other suitable algorithm for predicting output values based on input values.

In some embodiments, the regression model processes the features encoded in the feature vector by applying the parameters of the regression machine learning model to produce a model output vector. In some embodiments, the model output vector may be decoded to generate one or more numerical output values indicative of the range of demyelination. In some embodiments, the model output vector may include or may be decoded to reveal the output value(s) based on a modelled correlation between the feature vector and a target output. In some embodiments, the numerical output may represent the conduction slowing and/or a range of values of the conduction slowing.

In some embodiments, the parameters of the regression model may be trained based on known outputs. For example, the CMAP measurements may be paired with a target value or known value to form a training pair, such as a historical CMAP measurement of a patient in a study and an observed result and/or human annotated value representing a data point in the relationship between the historical CMAP measurement and conduction slowing. In some embodiments, the CMAP measurement may be provided to the regression model, e.g., encoded in a feature vector, to produce a predicted output value. In some embodiments, an optimizer associated with the regression model may then compare the predicted output value with the known output of a training pair including the historical CMAP measurement to determine an error of the predicted output value. In some embodiments, the optimizer may employ a loss function, such as, e.g., Hinge Loss, Multi-class SVM Loss, Cross Entropy Loss, Negative Log Likelihood, or other suitable classification loss function to determine the error of the predicted output value based on the known output.

In some embodiments, a neuroinflammatory biomarker activity measure can be determined from the biological sample of the patient. Thereafter, the biomarker activity measure can be compared against a predetermined biomarker activity threshold to determine whether the biomarker activity is above, equal, or below the predetermined biomarker activity threshold as shown at 109. As discussed above, an example of a predetermined biomarker activity threshold can be urine sPLA2 activity of 1371.93 pmol/min/ml however, other threshold value found to be statistically differentiator between patients with demyelinating neuropathy and patients not having demyelinating neuropathy can be equally used. For example, the threshold value may be any value in a range of between 500 and 2000 pmol/min/ml, in a range of between 600 and 2000 pmol/min/ml, in a range of between 700 and 2000 pmol/min/ml, in a range of between 800 and 2000 pmol/min/ml, in a range of between 800 and 2000 pmol/min/ml, in a range of between 900 and 2000 pmol/min/ml, in a range of between 1000 and 2000 pmol/min/ml, in a range of between 1100 and 2000 pmol/min/ml, in a range of between 1200 and 2000 pmol/min/ml, in a range of between 1300 and 2000 pmol/min/ml, in a range of between 1400 and 2000 pmol/min/ml, in a range of between 1500 and 2000 pmol/min/ml, in a range of between 500 and 1000 pmol/min/ml, in a range of between 500 and 1100 pmol/min/ml, in a range of between 500 and 1200 pmol/min/ml, in a range of between 500 and 1300 pmol/min/ml, in a range of between 500 and 1400 pmol/min/ml, in a range of between 500 and 1500 pmol/min/ml, in a range of between 500 and 1600 pmol/min/ml, in a range of between 500 and 1700 pmol/min/ml, in a range of between 500 and 1800 pmol/min/ml, in a range of between 500 and 1900 pmol/min/ml, in a range of between 1000 and 1500 pmol/min/ml.

In some embodiments, the patient can be identified as having a demyelinating neuropathy based on the conduction measure that is within the demyelinating range defined by the regression model and the neuroinflammatory biomarker activity measure of the patient that is above the predetermined threshold as shown at 111. Some examples of demyelinating neuropathy that can be identified include distal symmetrical polyneuropathy, chronic inflammatory demyelinating polyneuropathy, peripheral neuropathy, and other suitable types of demyelinating neuropathies.

In some embodiments, the patient that has been identified as having the demyelinating neuropathy can be administered a therapeutically effective amount of at least one agent used to treat the demyelinating neuropathy as shown at 113.

In some embodiments, when the patient is identified as having distal symmetric polyneuropathy, the patient can be administered a therapeutically effective amount of an agent. Such an agent can include several antidepressants, for example, duloxetine, venlafaxine, amitriptyline, and other suitable tricyclic drugs including amitriptyline, desipramine, nortriptyline, and other suitable tricyclic drugs. Likewise, the agent can include gabapentinoid antiepileptic drugs such as pregabalin, gabapentin, or other suitable gabapentinoid antiepileptic drugs.

In some embodiments, comorbidities and concurrent medications of patients identified as having distal symmetric polyneuropathy often favor one class or another (e.g., an antidepressant or a gabapentinoid) in an individual patient, and then the narrower choice within a class is influenced by the patient's age, preferences about dosing frequency, side effects, and cost/formulary considerations. Other agents that can be used to treat distal symmetric polyneuropathy include alpha-lipoic acid, valproic acid (e.g., taking 500 mg to 1200 mg daily), and carbamazepine.

In some embodiments, when a patient's comorbidities favor an antidepressant—a serotonin-norepinephrine reuptake inhibitor (SNRI; duloxetine or venlafaxine) agent can be administered to the patient identified as having distal symmetric polyneuropathy. In some other embodiments, when patient comorbidities favor a gabapentinoid pregabalin, gabapentin, or other gabapentinoid agent can be administered to the patient identified as having distal symmetric polyneuropathy.

In some embodiments, non-pharmacologic agents can be administered to patients identified as having distal symmetric polyneuropathy such non-pharmacologic agents can include capsaicin cream (e.g., 0.075 percent applied topically four times daily), lidocaine patches, and other suitable non-pharmacologic agents.

In some embodiments, when the patient is identified as having chronic inflammatory demyelinating polyneuropathy, patients may have a mild version of the disease with minimal impact on function and quality of life, in such cases no treatment may be required. However, most patients are impaired by the disorder and need treatment. The mainstays of therapy for CIDP are intravenous immune globulin (IVIG), glucocorticoids, plasma exchange and immunosuppressive drugs.

In some embodiments, regarding initial therapy and ongoing therapy, for treatment-naïve patients with CIDP who have active disease and related disability, an initial immune-modulating treatment can be administered using either IVIG, glucocorticoids, or plasma exchange. In some embodiments, the initial choice among these equally effective therapies may be influenced by disease severity, concurrent illness, venous access, treatment side effects, availability, and cost.

In some embodiments, for patients with severe and fulminant CIDP, a treatment with a rapid immune-modulating therapy such as IVIG, plasma exchange, or pulse high-dose glucocorticoids can be administered, rather than standard-dose daily glucocorticoids. In some embodiments, an initial treatment may be administered with IVIG (if available) or pulse glucocorticoids due to increased ease of administration relative to plasma exchange.

In some embodiments, for patients with CIDP where the goal is to achieve remission, pulse high-dose glucocorticoids may be more effective than IVIG or plasma exchange. A glucocorticoid course of up to eight weeks may be employed to decide whether there is a treatment response. Alternative immunosuppressive agents can be administered when glucocorticoids are contraindicated or considered too risky; choices include cyclosporine, methotrexate, mycophenolate, and azathioprine.

In some embodiments, regarding refractory disease, for patients with severe CIDP (e.g., unstable active disease with a progressive or relapsing course) who are refractory to treatment with IVIG, glucocorticoids, and plasma exchange, alternative immunosuppressant treatment options can be administered including cyclosporine, methotrexate, azathioprine, mycophenolate, rituximab, and cyclophosphamide. The choice among these is dependent on a number of factors that include side effect profiles of the alternative treatments, disease severity, age, and sex of patient, and concurrent medical problems.

In some embodiments, for patients with moderately severe CIDP, the patients may be administered a treatment the includes cyclosporine agent. In some embodiments, keeping plasma trough levels between 100 and 150 ng/mL may ensure a therapeutic response. Improvement with cyclosporine therapy may occur within three months. However, the side effect profile for cyclosporine may be greater than for methotrexate.

In some embodiments, another alternative treatment for CIDP patients with no contraindications may be oral methotrexate (e.g., 7.5 to 15 mg once a week). Patients treated with methotrexate may receive folic acid 1 mg daily, or leucovorin 2.5 mg weekly, to prevent hematologic and other side effects.

In some embodiments, pulse intravenous (IV) cyclophosphamide may be effective and may lead to long-term remission on CIDP patients. In some embodiments, dosing may be 1000 mg/m2 infused monthly for six months. However, the risks and side effects must be carefully considered.

In some embodiments, when the patient is identified as having chronic inflammatory demyelinating polyneuropathy, the patient may be administered a therapeutically effective amount of an agent. Such an agent can include intravenous immune globulin agents, subcutaneous immune globulin agents, and/or glucocorticoid agents or other suitable immune modulator agents.

In some embodiments, when the patient is identified as having chronic inflammatory demyelinating polyneuropathy, the patient can be administered a therapeutically effective amount of immune modulator agents including azathioprine, cyclophosphamide, cyclosporine, etanercept, interferon alpha-2a, interferon beta-1a, mycophenolate mofetil, methotrexate, rituximab, tacrolimus, or other suitable immune modulator agent.

In some embodiments, when the patient is identified as having peripheral neuropathy, the patient can be administered a therapeutically effective amount of an agent. Such an agent can include antidepressants such as nortriptyline, serotonin-norepinephrine reuptake inhibitors such as duloxetine hydrochloride, or other suitable type of antidepressant.

In some other embodiments, when the patient is identified as having peripheral neuropathy, the patient can be administered a therapeutically effective amount of an agent. Such an agent can be an anticonvulsant or antiepileptic agent including gabapentin, pregabalin, topiramate, lamotrigine, carbamazepine, oxcarbazepine, and other suitable anticonvulsant or antiepileptic agent. A patient identified as having peripheral neuropathy can likewise be administered a therapeutically effective amount of an opioid-based agent such as tapentadol or another opioid-based agent.

In addition, demyelinating neuropathy may be the result of a patient taking medications for other conditions. For example, various medications for, e.g., cardiovascular conditions, chemotherapeutics, antimicrobacterial agents, immunosuppressants, among other medications, may cause or otherwise contribute to neuropathy. Examples of neuropathy causing medications are provided below in Table 2.

TABLE 2
Examples of agents to that cause or contribute to demyelinating neuropathy.

| Agent/Group | Pathogenesis | Type of Neuropathy |
|---|---|---|
| Cardiovascular agents | | |
| Statins | | Predominantly sensory neuropathy |
| Chemotherapeutics | | |
| Vincristine | Dysfunction of cellular and | Predominantly sensory neuropathy |
| | axonal transport mediated by | |
| | microtubules. | |
| Docetaxel | Dysfunction of cellular and | Sensorimotor axonal neuropathy |
| | axonal transport mediated by | |
| | microtubules. | |
| Paclitaxel | Dysfunction of cellular and | Sensorimotor axonal neuropathy |
| | axonal transport mediated by | |
| | microtubules. | |
| Cisplatin | Irreversible binding to DNA, | Sensory neuropathy |
| | neuronal apoptosis. | |
| Oxaliplatin | Acute: Dysfunction of | Acute sensory symptoms and chronic |
| | voltage-dependent sodium | sensory neuropathy |
| | channels | |
| | Chronic: Irreversible binding | |
| | to DNA, neuronal apoptosis | |
| Bortezomid | | Painful, small fiber sensory |
| | | neuropathy |
| Thalidomide | | Predominantly sensory neuropathy |
| Antimicrobacterial | | |
| agents | | |
| Isoniazid | | Primarily sensory neuropathy |
| Ethambutol | | Optic neuropathy |
| Linezolid | | Sensory neuropathy |
| Metronidazole | | Predominantly sensory neuropathy |
| Nitrofurantoin | | Sensorimotor, primarily axonal large |
| | | and small fiber neuropathy |
| Immunosuppressants | | |
| TNF-a inhibitors | T-cell or induced | Axonal, sensory neuropathy, |
| | autoantibody attack against | mononeuropathy simplex or |
| | myelin, ischemic processes | multiplex |
| | or inhibition of axon | |
| | signaling. | |
| Leflunomide | | Primarily motor axonal neuropathy |
| NRTIs | | Primarily motor axonal neuropathy |

In some embodiments, during nerve conduction studies, an entire nerve is electrically activated. Motor responses may be recorded by the position of electrodes over muscles. The motor response may be recorded as the compound muscle action potential (CMAP). A CMAP is a type of electromyography. CMAP refers to a group of almost simultaneous action potentials from several muscle fibers in the same area evoked by stimulation of the supplying motor nerve and are recorded as one multipeaked summated action potential. The summated action potential has a characteristic amplitude, referred hereinafter as CMAP amplitude. CMAP amplitudes are roughly proportional to the number of axons conducting between stimulating and recording electrodes.

In some embodiments, subjects with diabetic DSP (e.g., N=90 subjects) and control subjects (e.g., N=46 subjects) may be recruited. The Inflammatory Neuropathy Cause and Treatment (INCAT) disability score is a measure of activity limitation. INCAT score, neurophysiological and urine sPA2 activity data may be collected from diabetic DSP subjects. Urine sPA2 activity may be collected from 46 control subjects. In some embodiments, electrodiagnostic testing may be performed in diabetic patients in a quiet room, with limb temperature maintained at or above 32° C. and using standard methods and distances. Nerve conduction study parameters including compound muscle action potential (CMAP), distal latency (DL), conduction velocity (CV) and F latency of the median, ulnar, tibial, and peroneal nerves may be collected. In some embodiments, the parameters can be likewise gathered from other body parts of the subjects.

In some embodiments, consecutive diabetic subject with abnormal nerve conduction studies may be included in the study and divided into 2 groups: diabetic subjects with DSP, who had at least one motor nerve with CV slowing in the American Academy of Neurology's (AAN) demyelinating range (without fulfilling the AAN criteria of primary acquired demyelination), were included in group A; and, diabetic subjects with DSP without nerve CV slowing in the AAN demyelinating range were included in group B.

In some embodiments, the diagnosis of diabetic DSP may be defined based on known history of diabetes mellitus, the presence of symmetric signs of distal weakness and sensory dysfunction and the absence or reduction of deep tendon reflexes. Subjects with presence/co-existence of other known causes of neuropathy (e.g., B12 deficiency, exposure to neurotoxic agents), use of anti-inflammatory drugs, presence of malignancy, or other causes of systemic inflammation and active infection may be excluded from the study.

In some embodiments, an initial group of (e.g., N=76 subjects) subject may be identified with chronic inflammatory demyelinating polyneuropathy (CIDP) using the AAN criteria for primary acquired demyelination. Motor nerve CV collected from the CIDP group may be used to develop regression equations that determine, for each CMAP amplitude, the range of slowing expected from a primary demyelinating polyneuropathy. Abnormal motor CV data from the median, ulnar, tibial, and peroneal nerves may be collected. For each motor nerve studied, the CMAP and CV may be measured. Using our normal laboratory values, the data may be converted to a percentage of lower limit of normal. Using regression analysis, equations that linked CV to distal CMAP amplitude of median, ulnar, peroneal, and tibial nerves in the 76 subjects with CIDP may be generated. The normalized value for each attribute of motor nerve conduction may be then expressed as a square root transformation, fourth root transformation, or Log 10 transformation to achieve the best linear relationship between CMAP amplitude and CV. The validity of the equations may be tested using a second group of subjects CIDP subjects (e.g., N=38 subjects). In some embodiments, the developed and validated equations that assessed the range of slowing in CIDP subjects may be used to study CV slowing in a group of subjects diagnosed with amyotrophic lateral sclerosis (ALS) (e.g., N=95 subjects) fulfilling the El Escorial World Federation of Neurology revised criteria for the diagnosis of ALS. (Brooks, B. R., et al., El Escorial revisited: revised criteria for the diagnosis of amyotrophic lateral sclerosis. Amyotroph Lateral Scler Other Motor Neuron Disord, 2000. 1(5): p. 293-9, which is incorporated herein by reference in its entirety. In some embodiments, the ALS group may be used as a negative control to determine the maximum number of motor nerves falling outside the regression analysis confidence intervals for each patient, beyond which a CV slowing from a potential demyelination is suspected. The regression equations may be used retrospectively to compare CV slowing in 219 diabetic DSP and 219 axonal non-diabetic DSP subjects.

In some embodiments, regression analysis may be also used to investigate CV slowing in the diabetic DSP subjects prospectively recruited to test for urine sPLA2 activity. In some embodiments, the urine biomarker sPLA2 activity may be performed using a random urine sample from the diabetic subjects and 46 control subjects. The assay may be optimized using an excess amount of substrate. In some embodiments, product formation in the presence of an excess amount of substrate may be linear during a measurement period (e.g., a 15 minute, 20 minute, 25 minute, 30 minute, 35 minute, 40 minute, 45 minute, 50 minute, 55 minute, 60 minute measurement period, etc.) such that the measured reaction rate may be proportional to urinary concentration of active sPLA2. The urine sPLA2 activity in healthy control subjects may be compared to that in diabetic DSP subjects.

In some embodiments, the urine samples may be centrifuged, e.g., at between 500 and 2000 rpm (e.g., at 1200 rpm) for between 1 and 10 minutes (e.g., 5 minutes). Each urine sample of 20 ul may be fractionated by 4-20% SDS-PAGE and then transferred to PVDF membrane. The blot may be probed with anti-NF-M/H and anti-NF-L antibodies, respectively. The positive samples may be repeated at least three times.

In some embodiments, data summary statistics may be performed using Microsoft® Office Excel 2016. SAS® Software (version 9.4) may be used to perform advanced statistical analysis of the data. Categorical variables may be summarized by their counts and percentages, and distribution of groups may be compared using Fisher's exact test or chi-square test. Z-test of proportions may be used to compare two proportions. Continuous variables may be summarized by their means and standard deviations [Mean (SD)] along with range, and the distribution of groups may be tested using t-test or analysis of variance as appropriate. In some embodiments, tests for statistical significance may be two-sided with a significance level of 0.05.

Exemplary regression models that may be utilized to assess the range of slowing in 76 CIDP patients are summarized in Table 3.

TABLE 3
Regression Models.

| Nerve | Conduction velocity | Distal latency | F Latency |
|---|---|---|---|
| Median | Y = 0.179 X + 2.463 +/− | Y = −0.177 X + 2.542 +/− | Y = −0.010 X + 2.243 +/− |
| | 0.2192 | 0.1663 | 0.09463 |
| | Y = Fourth root of CV | Y = Log₁₀ Value of DL | Y = Log₁₀ value of F |
| | X = Log₁₀ Value of | X = Log₁₀ Value of CMAP | X = Square root value of |
| | CMAP | | CMAP |
| Ulnar | Y = 2.579 X + 3.771 +/− | Y = −0.420 X + 4.227 +/− | Y = −2.263 X + 16.278 +/− |
| | 1.26189 | 0.2853 | 1.2635 |
| | Y = Square root of CV | Y = Fourth root of DL | Y = Square root of F |
| | X = Log₁₀ of CMAP | X = Log₁₀ of CMAP | X = Log₁₀ of CMAP |
| Tibial | Y = 0.532 X + 7.570 +/− | Y = −0.056 X + 158.617 +/− | Y = −0.003 X + 2.109 +/− |
| | 0.9983 | 48.3037 | 0.0413 |
| | Y = Square root of CV | Y = DL | Y = Log₁₀ value of F |
| | X = Log₁₀ value of | X = CMAP | X = Square root of |
| | CMAP | | CMAP |
| Peroneal | Y = 0.038 X + 2.817 +/− | Y = −0.033 X + 3.556 +/− | Y = −0.009 X + 2.082 +/− |
| | 0.2311 | 0.3057 | 0.0430 |
| | Y = Fourth root of CV | Y = Fourth root of DL | Y = Log₁₀ of F |
| | X = Log₁₀ Value of | X = Log₁₀ Value of CMAP | X = Log₁₀ of CMAP |
| | CMAP | | |

CMAP, compound muscle action potential; CV, conduction velocity; DL, distal latency; F; F latency.

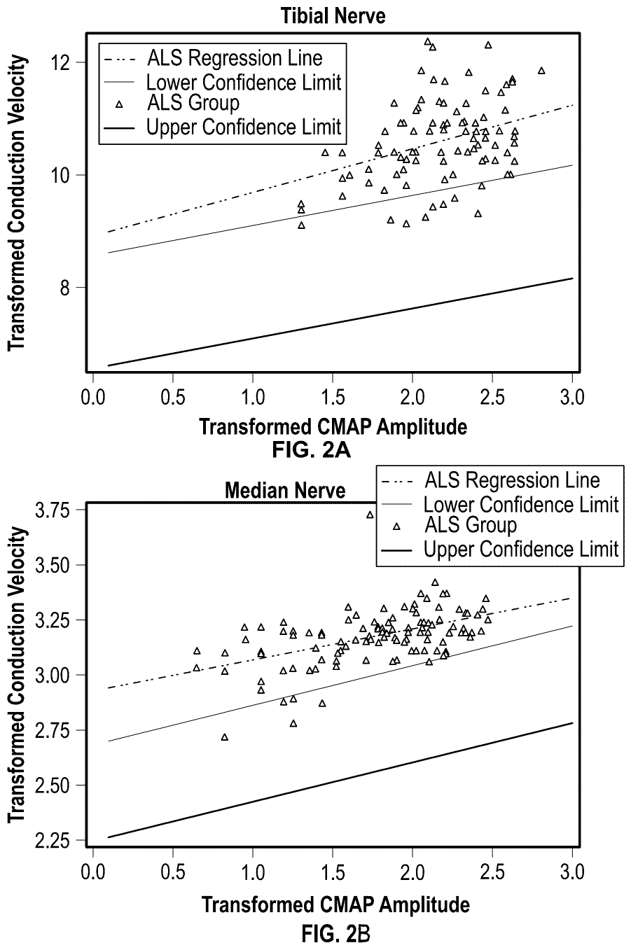

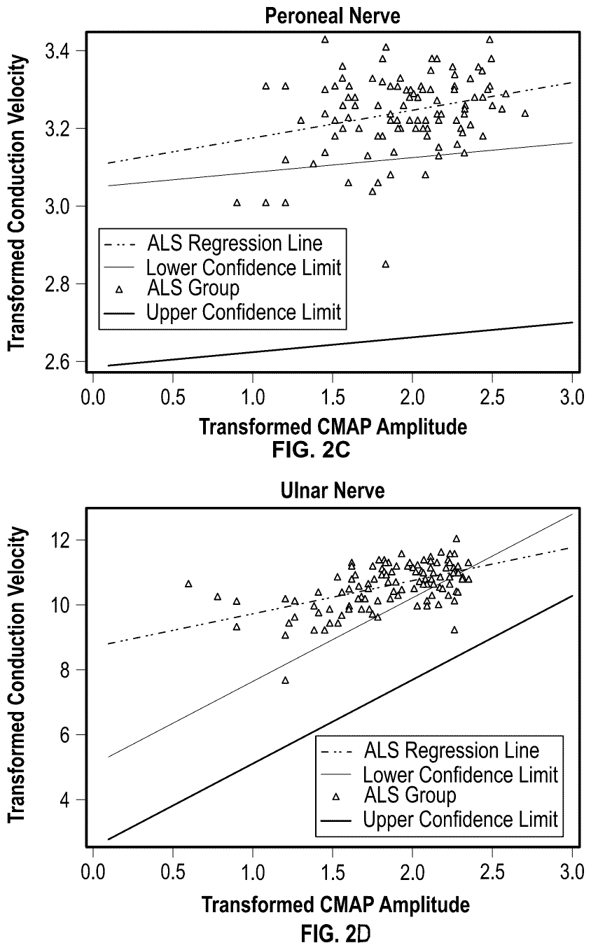

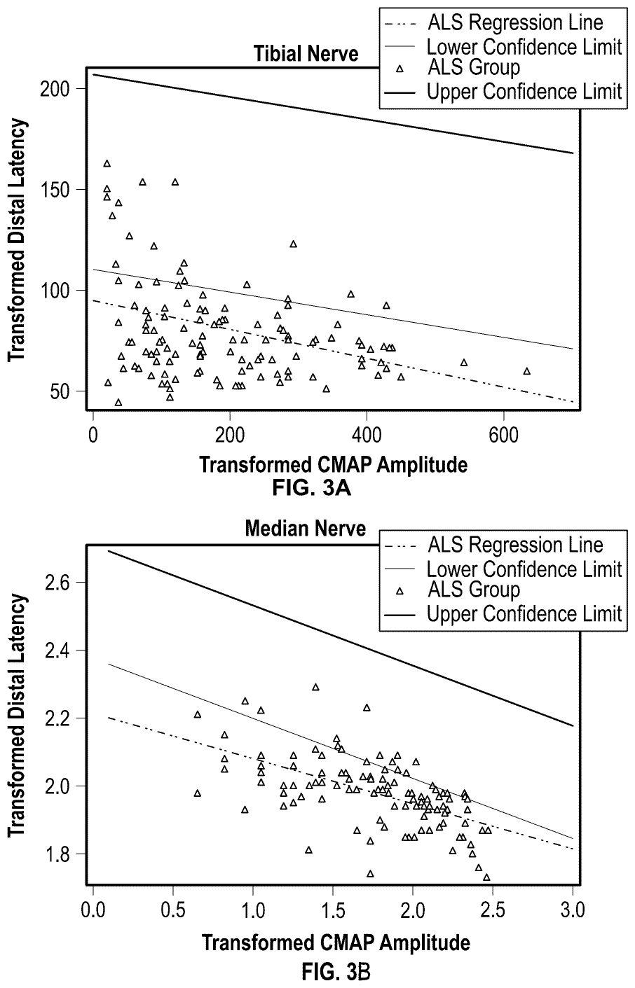

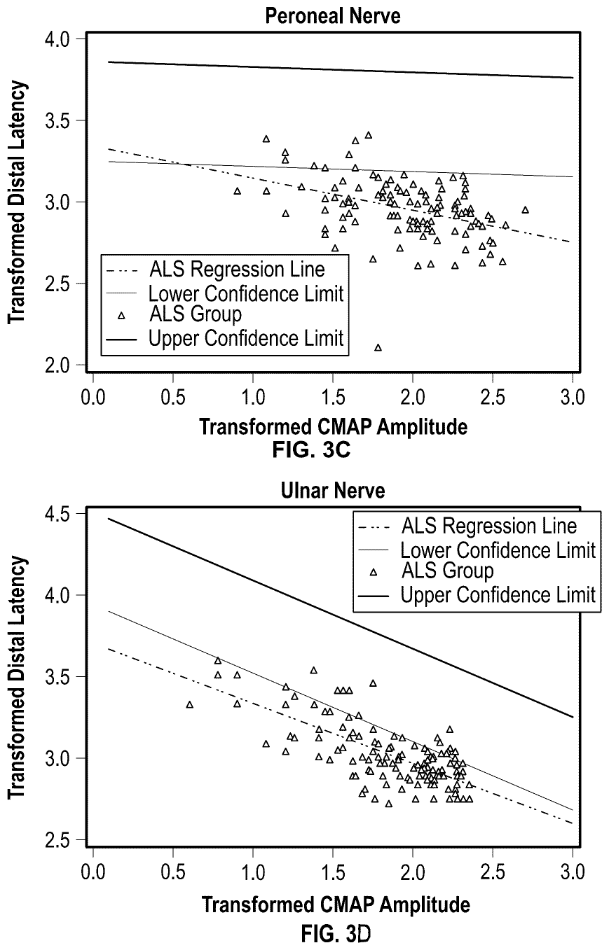

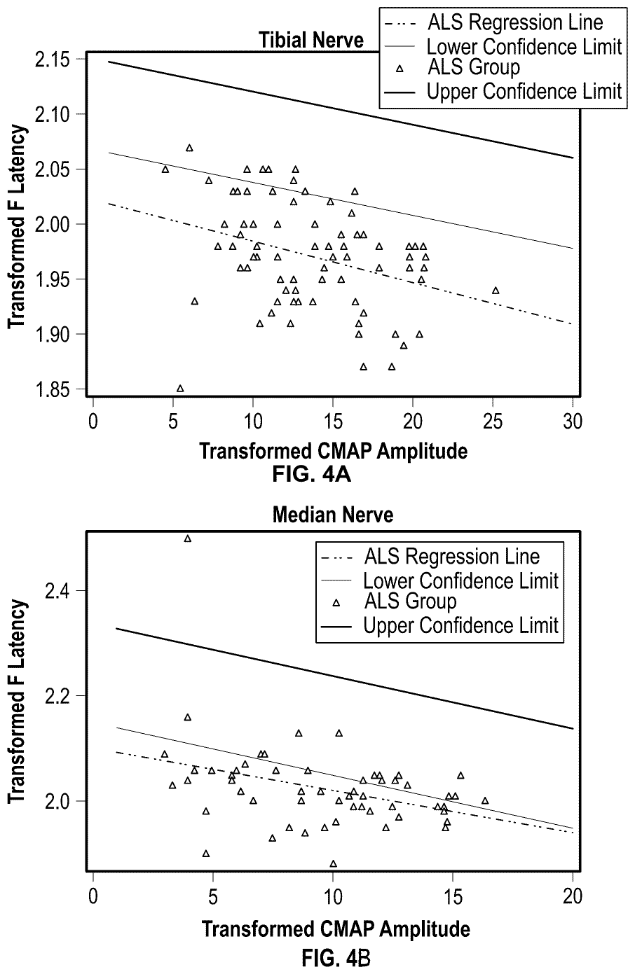

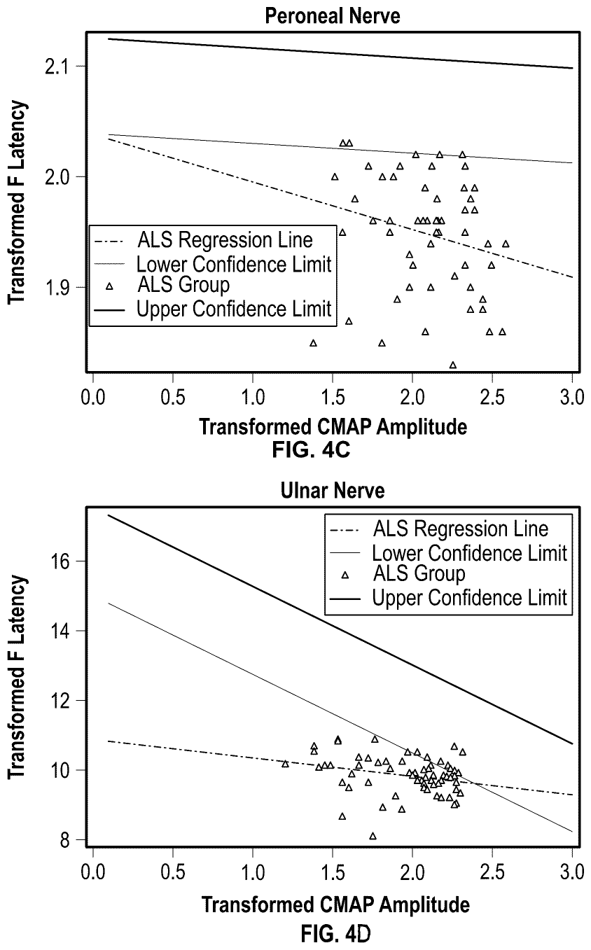

FIG. 2A, FIG. 2B, FIG. 2C and FIG. 2D illustrate converted data for CMAP amplitude versus conduction velocity for the tibial, peroneal, median, and ulnar nerve from a group of 95 subjects diagnosed with amyotrophic lateral sclerosis (ALS). FIG. 3A, FIG. 3B, FIG. 3C and FIG. 3D illustrate converted data for CMAP amplitude versus distal latency for the tibial, peroneal, median, and ulnar nerve from a group of 95 subjects diagnosed with amyotrophic lateral sclerosis (ALS). FIG. 4A, FIG. 4B, FIG. 4C and FIG. 4D illustrate converted data for CMAP amplitude versus F responses for the tibial, peroneal, median, and ulnar nerve from a group of 95 subjects diagnosed with amyotrophic lateral sclerosis (ALS).

In some embodiments, the conduction slowing in ALS results from a primary axonal loss. In some embodiments, the ALS group may be used as a negative control for a primary demyelination. Superimposed on FIGS. 2A-2D, FIGS. 3A-3D and FIGS. 4A-4D are the upper and lower limit confidence intervals derived from regression models, which may be from a group of CIDP subjects. In some embodiments, some, most or all of the data from ALS may be located outside the confidence intervals.

In some embodiments, the validity of the confidence intervals derived from the regression models may be performed by applying the confidence intervals to the electrophysiological data obtained from a group of subjects diagnosed with CIDP as per the AAN criteria. The regression models shown in Table 3, may be used to calculate the regression lines and 95%, 97%, 98%, 99%, 99.5%, etc. upper and lower confidence limits for CMAP amplitude versus DL, CV, and F latencies for the peroneal, tibial, median, and ulnar nerves. Subsequently, the data from the CIDP group of subjects may be used. In some embodiments, using a Wilcoxon Rank Sum test and the confidence intervals determined by the regression models, demonstrates that for all 12 equations, the data from the CIDP subject group may be within the developed confidence intervals and may not be statistically different from the data used to develop the regression models (e.g., 76 CIDP subjects) confirming the validity of the confidence intervals (e.g., all 12 p-values>0.05 significance level).

Table 1 above illustrates the range of conduction slowing in the demyelination range when the regression models were used with CMAP amplitude equal to the lower normal limit and 50% of the lower normal limit of the studied nerves.

### Example 1—Study Specific to Amyotrophic Lateral Sclerosis Patients

In some embodiments, the electrodiagnostic characteristics of patients diagnosed with ALS may be analyzed. The mean age of patients in ALS may be 58.5+11.70 years. There may be 58 men and 37 women. A total of 545 motor nerves may be studied: 133 median nerves, 139 ulnar nerves, 141 tibial nerves and 132 peroneal nerves after conversion of the raw data in the studied nerves to percentage of normal.

In some embodiments, the number of ALS patients with DL, CV, and F response slowing by the regression models' ranges criteria alone, and by the regression models' ranges criteria or AAN criteria combined are summarized in Table 4. No ALS subjects had more than 2 motor nerves with CV slowing in the AAN or regression equation ranges. In ALS subjects with 2 motor nerves or less in the demyelinating range, none of them had more than 4 DL or F responses in the demyelinating range either by regression equations or AAN criteria as shown on Table 4.

TABLE 4
The profile of conduction slowing in ALS patients.

| | DL | | | | |
|---|---|---|---|---|---|
| CV | DL0 | DL1 | DL2 | DL3 | F |
| CV0 | 31.6% (70.5%) | 6.3% (11.6%) | 2.1% (2.1%) | 2.1% (2.1%) | F0 |
| CV1 | 16.8% (18.9%) | 5.3% (5.3%) | 1.1% (2.1%) | 3.2% (3.2%) | F0 |
| CV2 | 1.1% (1.1%) | 1.1% (1.1%) | 1.1% (1.1%) | 0% (0%) | F0 |
| CV3 | 0% (0%) | 0% (0%) | 0% (0%) | 0% (0%) | F0 |
| CV0 | 6.3% (14.7%) | 2.1% (3.2%) | 0% (1.1%) | 0% (0%) | F1 |
| CV1 | 0% (0%) | 3.2% (3.2%) | 0% (0%) | 1.1% (1.1%) | F1 |
| CV2 | 0% (0%) | 0% (0%) | 0% (0%) | 1.1% (1.1%) | F1 |
| CV3 | 0% (0%) | 0% (0%) | 0% (0%) | 0% (0%) | F1 |
| CV0 | 0% (7.4%) | 0% (0%) | 1.1% (2.1%) | 1.1% (1.1%) | F2 |
| CV1 | 2.1% (3.2%) | 2.1% (2.1%) | 1.1% (1.1%) | 0% (0%) | F2 |
| CV2 | 0% (0%) | 0% (0%) | 0% (0%) | 0% (0%) | F2 |
| CV3 | 0% (0%) | 0% (0%) | 0% (0%) | 0% (0%) | F2 |
| CV0 | 0% (2.1%) | 0% (0%) | 2.1% (2.1%) | 1.1% (1.1%) | F3 |
| CV1 | 2.1% (2.1%) | 0% (1.1%) | 0% (0%) | 0% (0%) | F3 |
| CV2 | 0% (0%) | 0% (0%) | 0% (0%) | 0% (0%) | F3 |
| CV3 | 0% (0%) | 0% (0%) | 0% (0%) | 0% (0%) | F3 |

ALS; Amyotrophic lateral sclerosis; CV, conduction velocity; DL, distal latency; F; F latency.
CV0, CV1, CV2 and CV3 corresponds to patients with 0, 1, 2 and 3 motor nerves with CV in the equations' demyelinating range respectively.
DL0, DL1, DL2 and DL3 corresponds to patients with 0, 1, 2 and 3 motor nerves with DL in the equations' demyelinating range respectively.
F0, F1, F2 and F3 corresponds to patients with 0, 1, 2 and 3 motor nerves with F in the equations' demyelinating range respectively.
NOTE:
The values in parentheses represent percentage of patients in demyelination range by either AAN or regression equations' criteria.

### Example 2—Electrodiagnostic Testing of Diabetic Neuropathy and Axonal Non-Diabetic Neuropathy

In some embodiments, using electrodiagnostic data from CIDP patients, a regression analysis may be performed to develop confidence intervals that assess the range of conduction slowing from a primary demyelination. The regression analysis may be used to characterize conduction slowing in diabetic DSP group (e.g., N=219 subjects), axonal non-diabetic DSP group (e.g., N=219 subjects) and an ALS group (e.g., N=95 subjects).

In some embodiments, the study may be conducted using neurophysiological data obtained from 219 subjects diagnosed with diabetic DSP, 219 subjects with non-diabetic axonal DSP and 95 subjects diagnosed with ALS. The diagnosis of diabetic DSP may be defined based on known history of diabetes mellitus, the presence of symmetric signs of distal weakness and sensory dysfunction, and the absence or reduction of deep tendon reflexes. The diagnosis of axonal sensorimotor polyneuropathy may be based on the absence of history of diabetes, hyporeflexia or areflexia, the presence of distal weakness and sensory dysfunction, and the absence of electrodiagnostic signs of primary demyelination as per the AAN criteria.

In some embodiments, the demographic and neurophysiological data of diabetic DSP, non-diabetic DSP and ALS is summarized in Table 5A.

TABLE 5A
Demographic and neurophysiological profile of
diabetic DSP, non-diabetic DSP, and ALS groups.

| Parameter/ | DSP | Non-DSP | ALS | Overall |
|---|---|---|---|---|
| Groups | (n = 219) | (n = 219) | (n = 95) | p-value |
| Age (mean ± | 59.6 ± 12.20 | 58.4 ± 14.00 | 58.5 ± 11.76 | 0.9608 |
| SD) | | | | |
| Gender (% | 42.9% | 49.8% | 61.1% | 0.0122 |
| male) | | | | |

Distal Latency (ms)

| | | | | |
|---|---|---|---|---|
| Median | 4.7 ± 1.55 | 4.1 ± 1.15 | 4.4 ± 1.07 | <0.0001 |
| Ulnar | 3.3 ± 0.92 | 3.1 ± 0.73 | 3.5 ± 1.11 | <0.0001 |
| Tibial | 5.5 ± 1.4 | 5.4 ± 1.73 | 5.7 ± 1.82 | 0.0332 |
| Peroneal | 4.9 ± 1.58 | 4.7 ± 1.18 | 5.2 ± 1.29 | 0.1762 |

CMAP amplitude (mV)

| | | | | |
|---|---|---|---|---|
| Median | 6.6 ± 2.95 | 6.8 ± 2.99 | 4.0 ± 3.28 | <0.0001 |
| Ulnar | 6.8 ± 3.01 | 7.4 ± 2.91 | 4.7 ± 2.94 | <0.0001 |
| Tibial | 4.2 ± 3.16 | 5.0 ± 3.17 | 4.9 ± 3.21 | 0.0005 |
| Peroneal | 3.4 ± 2.18 | 3.6 ± 2.25 | 3.0 ± 2.33 | <0.0001 |

Conduction velocity (m/s)

| | | | | |
|---|---|---|---|---|
| Median | 46.1 ± 7.44 | 49.5 ± 6.14 | 50.2 ± 8.15 | <0.0001 |
| Ulnar | 48.2 ± 8.14 | 52.2 ± 7.63 | 53.1 ± 9.02 | <0.0001 |
| Tibial | 38.1 ± 7.84 | 40.7 ± 5.9 | 41.6 ± 5.84 | <0.0001 |
| Peroneal | 40.0 ± 7.45 | 42.8 ± 6.78 | 43.0 ± 5.65 | 0.0002 |

F response (converted percentages)

| | | | | |
|---|---|---|---|---|
| Median | 108.5 ± 16.86 | 106.6 ± 17.98 | 107.7 ± 30.02 | 0.5002 |
| Ulnar | 102.6 ± 16.54 | 99.5 ± 11.68 | 97.1 ± 10.21 | 0.0015 |
| Tibial | 95.3 ± 18.30 | 95.3 ± 14.72 | 94.1 ± 10.24 | 0.8037 |
| Peroneal | 91.6 ± 17.11 | 88.6 ± 15.28 | 88.3 ± 11.31 | 0.1076 |

DSP, Distal symmetrical polyneuropathy; ALS; Amyotrophic lateral sclerosis; CMAP, compound muscle action potential; CV, conduction velocity; DL, distal latency; F; F latency.

In some embodiments, there may be no difference of the mean age distribution of patients between the three study groups shown in Table 5A.

In some embodiments, the number of motor nerves with CMAP amplitude below lower limit of normal may be higher in the ALS group compared to the diabetic DSP and non-diabetic DSP respectively (e.g., 50.9% versus 36.2% versus 29.7%, p<0.0001).

In some embodiments, the mean CV may be statistically lower in diabetic DSP than in ALS and non-diabetic DSP groups for all four tested nerves as shown in Table 5A. The number of motor nerves with CV below 80% and 70% of lower limit of normal may be higher in the diabetic DSP group (e.g., 20.0% and 13.2%) compared to non-diabetic DSP group (e.g., 9.0% and 6.7%) and ALS group (e.g., 8.0% and 7.4%), p<0.0001.

In some embodiments, the Mean F response latency may be more prolonged in diabetic DSP group than in ALS and non-diabetic groups for ulnar nerve (e.g., p<0.05). There are more motor nerves with abnormal F responses in the diabetic DSP group compared to the ALS and non-diabetic group (e.g., p<0.05). Furthermore, the number of F responses with prolonged latency more than 120% and 150% may be higher in the diabetic DSP group (e.g., 8.7% and 0.7%), non-diabetic DSP group (e.g., 6.0% and 0.2%) and ALS group (e.g., 1.9% and 0.3%), p<0.0001.

In some embodiments, the Mean DL may be more prolonged in diabetic DSP group compared to non-diabetic DSP group for median, ulnar, and tibial nerves (e.g., p<0.05, shown in Table 5A). There are more motor nerves with prolonged DL in the diabetic DSP group compared to non-diabetic DSP group (e.g., 19.7% versus 13.7%, p<0.0001).

In some embodiments, the number of absent motor responses in diabetic DSP group may be higher than the ALS and non-diabetic DSP groups, respectively (e.g., 8.9% versus 6.1% versus 5.7%, p<0.0001).

In some embodiments, there are more motor nerves with reduced CV, prolonged corresponding distal latencies with abnormal corresponding F responses in the diabetic group compared to the ALS and the non-diabetic DSP group indicative of more diffuse slowing affecting proximal, middle, and distal nerve segments in the diabetic DSP patients (e.g., p<0.0001).

TABLE 5B
Conduction slowing in the diabetic DSP, non-diabetic DSP, and ALS groups

| | CV | CV | | | | | F | F | |
|---|---|---|---|---|---|---|---|---|---|
| | slowing | slowing in | | DL in | DL in | | slowing | slowing in | |
| | in AAN | equations | | AAN | equations | | in AAN | equations | |
| Groups | range | range | p-value | range | range | p-value | range | range | p-value |
| Diabetic DSP | 16.9% | 36.2% | <0.0001 | 4.3% | 21.2% | <0.0001 | 24.5% | 48.6% | <0.0001 |
| Non-diabetic DSP | 2.5% | 22.7% | <0.0001 | 2.8% | 15.9% | <0.0001 | 11.6% | 36.7% | <0.0001 |
| ALS | 1.4% | 9.4% | <0.0001 | 2.9% | 11.7% | <0.0001 | 15.8% | 31.3% | <0.0001 |

DSP, Distal symmetrical polyneuropathy; ALS; Amyotrophic lateral sclerosis; CMAP, compound muscle action potential; CV, conduction velocity; DL, distal latency; F; F latency.

In some embodiments, Table 5B shows that the number of motor nerves with CV, DL and F latencies fulfilling regression equations criteria for demyelination is higher than the number of motor nerves fulfilling the AAN criteria for demyelination in all studied groups. Furthermore, the number of motor nerves with CV and DL fulfilling regression equations' criteria for demyelination or AAN criteria for demyelination is higher in the diabetic DSP group compared to non-diabetic DSP and ALS groups.

In some embodiments, in the diabetic DSP group, the number of patients with at least one motor nerve with CV slowing in the demyelinating range by AAN criteria and regression equations ranges higher in the diabetic DSP group (e.g., 32.0% and 84.0%) compared to non-diabetic DSP group (e.g., 11.9% and 69.4%) and to ALS group (e.g., 7.4% and 44.2%), p<0.0001.

In some embodiments, no patients in the ALS group have more than two motor nerves with CV responses in the demyelination range either by the equations or AAN criteria. There are higher number of patients fulfilling the above criteria in the diabetic DSP group compared to the axonal non-diabetic group (e.g., 47.0% versus 23.3%; p<0.0001). Furthermore, there are more patients who had more than two motor nerves with CV slowing in the demyelination range by either the equations or AAN criteria with a least one motor nerve with corresponding F response in the demyelinating range by AAN criteria in the diabetic DSP group compared to the axonal non-diabetic group (e.g., 20.5% versus 7.8%, p=0.0001).

In some embodiments, among the patients who may have at least one motor nerve with CV slowing in AAN demyelinating range, there are more patients with more than 2 motor nerves with CV in the demyelinating range (e.g., either by AAN or regression equations criteria) in the diabetic DSP group compared to the axonal non-diabetic group (e.g., 32.0% versus 7.8%; p<0.0001). Furthermore, there may be more patients fulfilling the above criteria with at least one motor nerve with F response in the demyelinating range by AAN criteria (e.g., corresponding to a motor nerve with CV in the demyelinating range) in the diabetic DSP group compared to the axonal non-diabetic group (e.g., 28.8% versus 4.1%; p<0.0001).

In some embodiments, in the diabetic subgroup with at least one nerve with CV slowing in the AAN demyelination range the estimate of likelihood to have more than 2 motor nerves with CV slowing in the demyelination range by AAN or regression equations criteria is higher in the diabetic DSP group (e.g., 0.73) compared to non-diabetic DSP group (e.g., 0.52); p<0.05.

TABLE 6
Regression analysis of amplitude-dependent variation in distal latency, conduction
velocity and F response for diabetic DSP, non-diabetic DSP, and ALS groups.

| | | | | | |
|---|---|---|---|---|---|
| Change | Change | Change | Change | Change | Change |
| in DL | in CV | in F | in DL | in CV | in F |

DSP Group

| | | | | | | | |
|---|---|---|---|---|---|---|---|
| Median | 377 | 372 | 320 | Tibial | 318 | 314 | 259 |
| nerve (n) | | | | nerve (n) | | | |
| Intercept | 2.524 | 2.671 | 2.119 | Intercept | 89.087 | 7.738 | 2.000 |
| Slope | −0.248 | 0.208 | −0.008 | Slope | −0.064 | 1.138 | −0.002 |
| r^{a} | −0.539 | 0.437 | −0.287 | r^{a} | −0.431 | 0.437 | −0.114 |
| P-value | <0.0001 | <0.0001 | <0.0001 | P-value | <0.0001 | <0.0001 | 0.0084 |

Non-diabetic DSP Group

| | | | | | | | |
|---|---|---|---|---|---|---|---|
| Median | 336 | 332 | 282 | Tibial | 364 | 359 | 297 |
| nerve (n) | | | | nerve (n) | | | |
| Intercept | 2.381 | 2.777 | 2.083 | Intercept | 94.260 | 8.525 | 2.049 |
| Slope | −0.203 | 0.182 | −0.005 | Slope | −0.087 | 0.881 | −0.005 |
| r^{a} | −0.467 | 0.445 | −0.187 | r^{a} | −0.547 | 0.415 | −0.367 |
| P-value | <0.0001 | <0.0001 | 0.0126 | P-value | <0.0001 | <0.0001 | <0.0001 |

ALS Group

| | | | | | | | |
|---|---|---|---|---|---|---|---|
| Median | 122 | 121 | 62 | Tibial | 135 | 104 | 88 |
| nerve (n) | | | | nerve (n) | | | |
| Intercept | 2.175 | 2.925 | 2.102 | Intercept | 97.573 | 8.926 | 2.020 |
| Slope | −0.133 | 0.141 | −0.008 | Slope | −0.071 | 0.767 | −0.004 |
| r^{a} | −0.601 | 0.494 | −0.357 | r^{a} | −0.352 | 0.369 | −0.362 |
| P-value | <0.0001 | <0.0001 | 0.004 | P-value | <0.0001 | 0.0001 | 0.005 |

DSP, Distal symmetrical polyneuropathy; CV, conduction velocity; DL, distal latency; n, number of motor nerves.
^{a}Pearson correlation coefficient.

In some embodiments, correlation and regression analyses may be conducted for the associations between CMAP amplitude and CV, DL, and F response in the diabetic DSP, non-diabetic DSP and ALS groups, and the Pearson correlation coefficients as well as slopes and intercepts from regression are summarized in Table 6 above. Pearson correlation coefficients indicate correlation between the CMAP amplitude and CV, DL, and F responses in all studied groups.

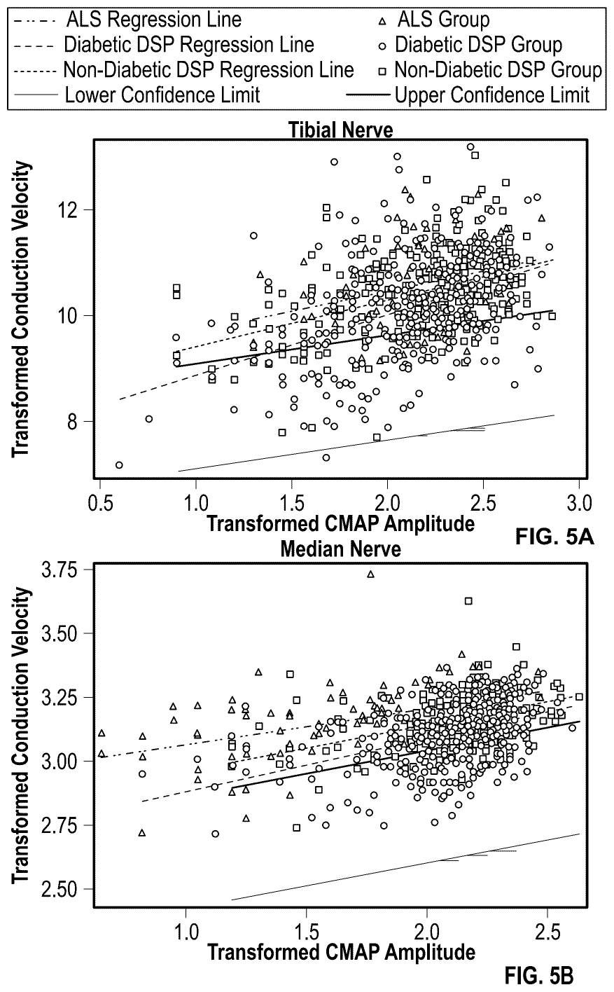

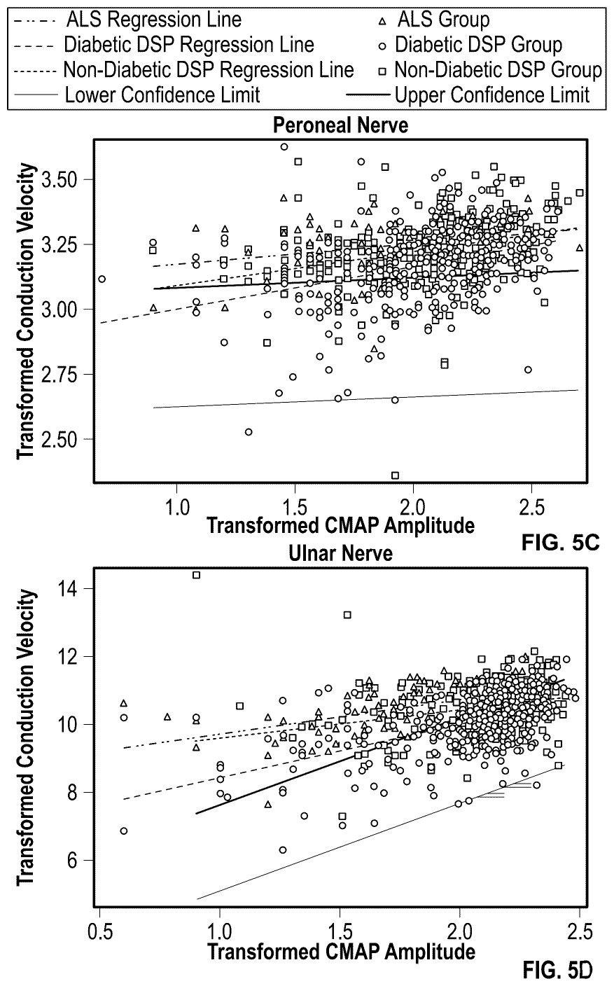

In some embodiments, the in addition or in the alternative, analysis of covariance (ANCOVA) may be conducted to assess the association of CMAP amplitude (as independent variable) with CV (as dependent variables) for the three groups. Regression coefficient of Y-intercept may be lower in the diabetic DSP group compared to the non-diabetic DSP group and to the ALS group that cannot be explained by the difference in the slopes of these groups (e.g., p<0.05, FIG. 5A, FIG. 5B, FIG. 5C and FIG. 5D).

FIG. 5A, FIG. 5B, FIG. 5C and FIG. 5D illustrate regression plots of converted normalized CV (y-axis, dependent variable) and converted normalized CMAP amplitude (x-axis, independent variable) for the diabetic DSP, non-diabetic DSP, and ALS groups.

As shown in FIG. 5A-5D there may be a difference in the slopes of the CV for the median, peroneal, and ulnar nerves in the diabetic DSP group compared to the ALS group (e.g., p<0.05), but not in tibial nerve. Similar findings can be observed for the ulnar nerve in the diabetic DSP group compared to the non-diabetic DSP group (e.g., p<0.05). In some embodiments, these findings may demonstrate that for the same CMAP amplitude, the corresponding CV may be lower in the diabetic group compared to the ALS group in median, peroneal and ulnar nerves, and to non-diabetic DSP for the ulnar nerve.

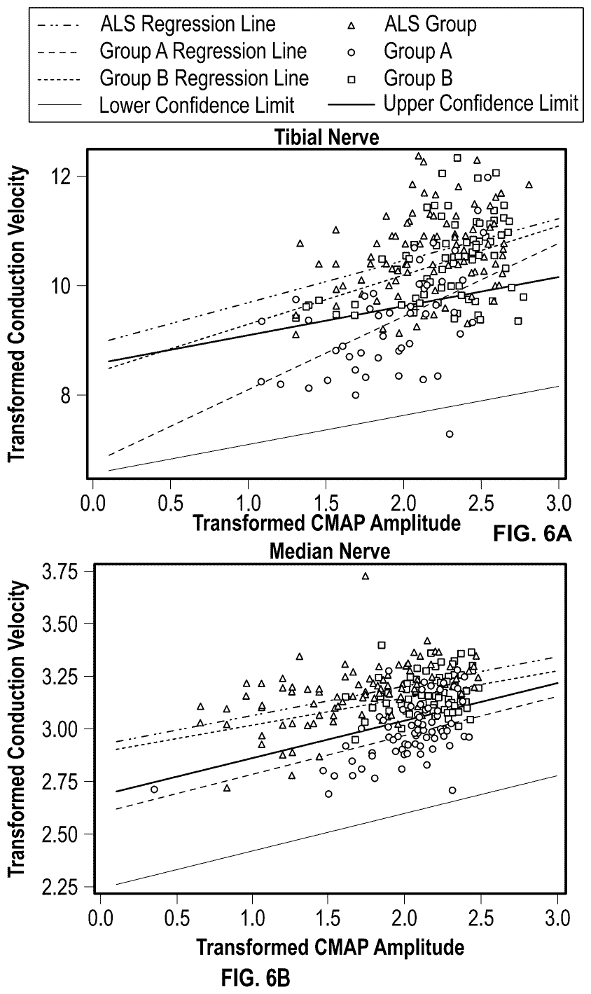

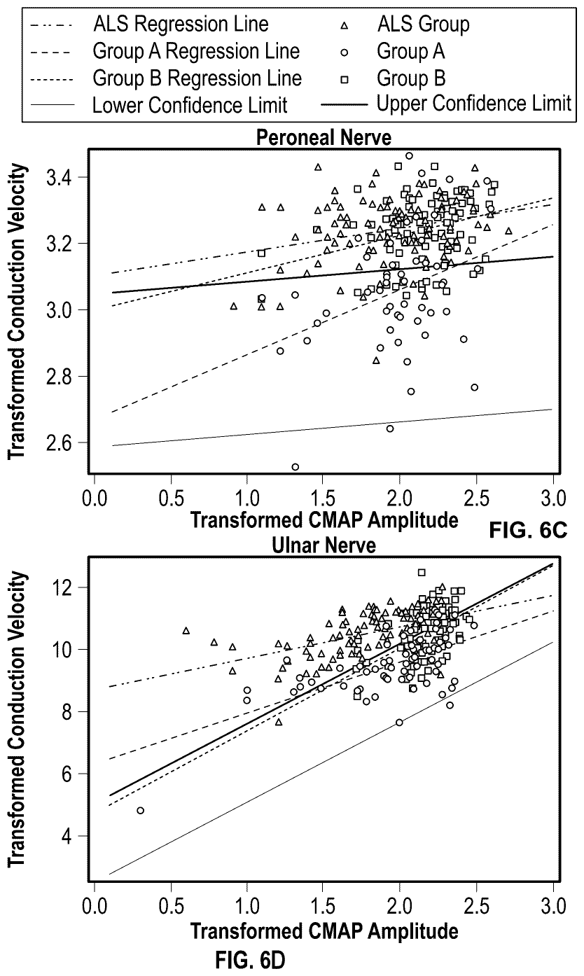

In FIG. 6A, FIG. 6B, FIG. 6C and FIG. 6D the raw CMAP amplitudes and CV data are expressed as fraction of lower limit of normal then transformed to Log 10 for CMAP amplitude, square root for CV of the tibial and ulnar nerves, fourth root for CV of median and peroneal nerves to achieve a linear relationship between CMAP amplitude and CV measurements. Additionally, CMAP amplitude-independent CV slowing may be observed in all motor nerves in the diabetic DSP relative to non-diabetic DSP and ALS groups: the plots shown in FIG. 6A, FIG. 6B, FIG. 6C and FIG. 6D demonstrate correlation between CMAP amplitude and CV in all diabetic DSP groups and the non-diabetic groups for the studied motor nerves. Regression coefficient of Y-intercept may be lower in the diabetic DSP group compared to the non-diabetic DSP group and to the ALS group that cannot be explained by the difference in the slopes of these groups. In FIG. 6A, FIG. 6B, FIG. 6C and FIG. 6D the diabetic DSP data points are depicted by closed triangles, the non-diabetic DSP data points are depicted by open triangles, and ALS data points are depicted by open circles. Regression lines for diabetic DSP, non-diabetic DSP and ALS are depicted by a solid bold line, solid line, and broken line respectively.

In some embodiments, as shown in the data above, the conduction slowing in diabetic DSP may be beyond from what is expected from a pure axonal loss and a primary or secondary demyelination is contributing to the electrodiagnostic findings in these patients. Furthermore, the presence of at least one motor nerve with CV in the AAN demyelinating range may increase the likelihood of having other motor nerves with abnormal conduction slowing beyond what is expected from a pure axonal loss.

In some embodiments, the conduction slowing in diabetic DSP may be beyond from what expected from a pure axonal loss and a primary or secondary demyelination is contributing to the electrodiagnostic findings.

In some embodiments, the presence of more than two motor nerves with conduction slowing in the AAN or regression equations confidence intervals may be indicative of demyelination as a contributing factor for the observed conduction slowing.

In some embodiments, in the diabetic DSP group, the presence of at least one motor nerve with CV slowing in the AAN range may demonstrate the contribution of primary or secondary demyelinating process, not enough severe to fulfill the AAN criteria for primary demyelination. This is supported by the increase in the likelihood to have more than two motor nerves with conduction slowing in the AAN or regression equations confidence intervals in diabetic DSP patients with at least one motor nerve with CV slowing in the AAN demyelinating range compared to ALS and non-diabetic neuropathy groups.

In some embodiments, the loss of large, myelinated fibers is the major contributing factor for conduction slowing in the diabetic DSP group, axonal non-diabetic neuropathy group, and ALS group. This is illustrated by CMAP amplitude dependent slowing of CV, DL, and F responses in most studied nerves in distal, intermediate, and proximal motor nerve segments. However, the coefficient for the slope of CMAP versus CV in the multiple regression analyses may be different in the diabetic DSP group compared to the ALS group: CV may be uniformly lower overall ranges of CMAP amplitude in the diabetic group compared to the ALS group. Furthermore, there are lower regression coefficients of the Y intercept of CV in all studied nerves in the diabetic DSP group compared to ALS and axonal non-diabetic groups. These findings indicate that there is a superimposed CMAP independent CV of intermediate nerve segments.

In some embodiments, the regression models may be a valuable tool to identify and characterize conduction slowing beyond what is expected from a pure axonal loss.

In some embodiments, to differentiate between CIDP, a potentially treatable disorder, and axonal polyneuropathy with loss of fast conducting axons, an irreversible process, the AAN sets electrodiagnostic criteria with high specificity and low sensitivity. Conduction slowing seldom fulfilling the AAN criteria for CIDP has been observed in diabetic DSP. Although a difference in conduction slowing may be found between diabetic DSP patients and non-diabetic purely axonal neuropathies, there may be overlap between the electrophysiologic findings in the two groups. This overlap may be caused by the difficulties in documenting demyelination in diabetic DSP, especially in mild or early cases in which enough demyelination has not yet occurred, in advanced cases in which secondary axon loss precludes detection of demyelination, and in diabetic patients with demyelination confined mainly to proximal nerve segments that are difficult to study with conventional electrodiagnostic testing.

In some embodiments, the regression models with confidence intervals derived from a well-defined cohort of patients with CIDP, may be more sensitive than the AAN criteria in identifying conduction slowing beyond what is expected from a pure axonal loss.

In some embodiments, the methods disclosed herein provide a better characterization of conduction slowing in diabetic DSP a problem that is of utmost importance to identify the patients with reversible diabetic neuropathy and determine the appropriate timing to implement therapeutic interventions and avoid the progression to irreversible axonal loss.

In some embodiments, the regression models presented herein define for each CMAP amplitude, a confidence interval for conduction slowing related to primary demyelinating process. When these regression models are used in conjunction with electrodiagnostic data obtained from a diabetic DSP group, non-diabetic DSP groups, and ALS groups, the regression models demonstrated different coefficient for the slope of CMAP versus CV and lower Y intercept of CV in the diabetic DSP compared to other groups supporting a conduction slowing unrelated to a pure axonal loss. In some embodiments, when the regression models are applied in conjunction to electrodiagnostic data from ALS group, there may be no patient with more than 2 motor nerve with CV slowing in the AAN or equations ranges, setting up a minimum number of motor nerves for each diabetic DSP subject with CV slowing fulfilling the regression equations beyond a point in which the presence of demyelinating process may be considered. Additionally, the likelihood of having more than 2 motor nerves with conduction slowing in AAN or regression models may be higher in the diabetic DSP with at least one nerve with CV slowing in the AAN demyelination range compared to non-diabetic DSP illustrating the probability of diffuse demyelinating process, not enough severe to fulfill the AAN criteria for primary demyelination, causing the conduction slowing in the diabetes group. Conventional electrodiagnostic testing as well AAN criteria for primary demyelination may be silent when the process is mild or focal.

In some embodiments, the presence of more than two motor nerves with CV in the AAN or regression models ranges may be indicative of the contribution of demyelinating process. The presented regression models may have more sensitivity in detecting mild demyelination than AAN criteria, an overlap with a mild to moderate axonal loss relatively preserving CMAP amplitude remained possible. In some embodiments, a portion of non-diabetic axonal DSP may have more than two motor nerves with CV in the demyelinating range, whereas there may be no ALS patient with such CV slowing. In some embodiments, the overlap may reflect the variability in conduction slowing in CIDP electrodiagnostic data from which the regression equations may be derived. The overlap may be minimized, e.g., to 4.1% or less, or 5% or less, when prolonged F response is included in the demyelinating range to further characterize the diabetic DSP and axonal non-diabetic group. The presence of higher incidence an additional prolonged F response to CV in the demyelinating range in the diabetic DSP group may be an indicator of more diffuse demyelinating affecting the proximal segment of the motor nerve in addition to its intermediate segment.

In some embodiments, given an absence of specific serum biomarker for CIDP, the use of the regression models in diabetic DSP subjects not or partially fulfilling the AAN criteria for primary demyelination, in whom there is atypical or stepwise progressive, or recurrent symmetric proximal and distal weakness, may improve the ability to characterize mild demyelination in diabetic DSP. Furthermore, because the regression models may be designed to achieve the best linear relationship between CMAP amplitude of the corresponding CV, the resulting confidence interval may not capture severe CV in the AAN demyelinating range. Therefore, combining the regression equations with the AAN criteria for demyelination and to an adequate clinical assessment may improve the sensitivity and specificity of the regression models to identify conduction slowing not resulting from a pure axonal loss that could be amenable to therapeutic interventions.

In some embodiments, the regression models of CMAP amplitude may identify a conduction slowing in diabetic DSP beyond what is expected from an axonal loss observed in ALS and axonal non-diabetic DSP groups. The regression models may be more sensitive than conventional electrodiagnostic testing to detect conduction slowing compatible with the diagnosis of mild demyelination.

### Example 3—Regression Analysis Combined with Urine Biomarker

Table 7 illustrates demographic, clinical, and electrodiagnostic summary statistic from subjects in diabetic DSP group A (e.g., N=37 subjects) subjects from group A have at least one motor nerve with CV slowing fulfilling the AAN criteria for acquired demyelination and subjects in group B (e.g., N=53 subjects) subjects in group B have no motor nerves with CV slowing fulfilling the AAN criteria for acquired demyelination.

In some embodiments, as shown in Table 7, there may be no difference observed in age and gender in the two diabetic DSP groups (e.g., Table 7). Mean INCAT score may be higher in the group A compared to group B (e.g., 2.3 versus 1.2, p<0.05 as shown in this example). Mean CMAP amplitude and CV may be lower in all studied motor nerves in group A compared to group B (e.g., Table 7).

TABLE 7
Demographic and electrodiagnostic characteristics of diabetic DSP patients.

| | Group A: Diabetic | Group B: Diabetic | |
|---|---|---|---|
| | patients with CV | patients without CV | |
| | slowing in the AAN | slowing in the AAN | |
| | range | range | |
| | (n = 37) | (n = 53) | p-value |
| Age (years) | 57.7 ± 10.84 | 57.5 ± 11.26 | 0.9261 |
| Sex | | | |
| Male % | 45.9% | 26.4% | 0.0722 |
| Female % | 54.1% | 73.6% | |
| Mean INCAT score | 2.3 ± 2.09 | 1.2 ± 1.11 | 0.0025 |
| Mean Urine sPLA2 activity in | 1328.3 ± 1274.21 | 673.8 ± 576.93 | 0.0014 |
| pmol/min/ml) | | | |
| Number of patients with | 15 (40.5%) | 8 (15.1%) | 0.0129 |
| increased urine sPLA2 (mean of | | | |
| controls ± 2SD, >1371.93 | | | |
| pmol/min/ml | | | |
| CMAP distal amplitude (mV) | | | |
| Tibial nerve | 2.9 ± 2.13 | 5.9 ± 3.16 | <0.0001 |
| Peroneal nerve | 3.0 ± 2.08 | 4.1 ± 2.13 | 0.0055 |
| Median nerve | 5.2 ± 2.41 | 7.4 ± 2.75 | <0.0001 |
| Ulnar nerve | 5.8 ± 3.22 | 8.1 ± 2.33 | <0.0001 |
| Conduction Velocity (m/s) | | | |
| Tibial nerve | 32.6 ± 6.81 | 40.6 ± 5.49 | <0.0001 |
| Peroneal nerve | 34.8 ± 8.25 | 43.2 ± 5.36 | <0.0001 |
| Median nerve | 38.6 ± 6.54 | 49.9 ± 5.75 | <0.0001 |
| Ulnar nerve | 43.0 ± 8.16 | 52.5 ± 7.54 | <0.0001 |

DSP, Distal symmetrical polyneuropathy; CMAP, compound muscle action potential; CV, conduction velocity.

FIG. 6A and FIG. 6B illustrate the confidence intervals that may assess the range of CV slowing in the median, ulnar, tibial, and peroneal motor nerve in 76 subjects with CIDP. The validity of the confidence intervals derived from regression models may be confirmed using a control group of 38 subjects diagnosed with CIDP as per the AAN criteria. Using Wilcoxon Rank Sum test, it is demonstrated that all 4-regression model data from the control group may be within that confidence intervals determined by the first group of 76 CIDP patients confirming the validity of the confidence intervals (all 12 p-values may be, e.g., >0.05 significance level).

In FIG. 6A and FIG. 6B raw CMAP amplitudes and CV data are expressed as fraction of lower limit of normal and then transformed to Log 10 for CMAP amplitude, square root for CV of the tibial and ulnar nerves, fourth root for CV of median and peroneal nerves to achieve a more linear relationship between CMAP amplitude and CV measurements. ALS group data points are depicted by hollow triangles, diabetic Group A data points by filled circles and diabetic Group B data points by hollow squares. Regression lines for ALS, diabetic Group A and diabetic Group B are depicted by a dash-double-dot line, a dash line, and a short-dash line respectively.

In some embodiments, the regression models may be implemented to achieve the best linear relationship between the CMAP amplitude of the corresponding CV, the resulting confidence interval may exclude some severe conduction slowing. However, all severe CV slowing outside the equations' confidence intervals may be in the AAN demyelinating range. In some embodiments, the regression confidence intervals may be used in combination with AAN criteria for primary demyelination to capture conduction slowing not severe enough to fulfil the AAN demyelination criteria.

In some embodiments, the subjects in the ALS group may be unlikely to have more than 2 motor nerves with CV slowing by the AAN criteria or regression equations' confidence intervals as shown in FIG. 5A and FIG. 5B. Therefore, the presence of more than two motor nerves with CV slowing by the AAN or regression model ranges may be indicative of CV slowing beyond what is expected from a pure axonal loss and could be related to a demyelinating process.

In some embodiments, a prospectively recruited group of diabetic DSP subjects may have a higher number of patients with more than 2 motor nerves with CV slowing in the AAN or regression equations ranges in group A compared to group B (see, e.g., 83.8% versus 28.3%, p<0.0001, FIG. 5A and FIG. 5B). Furthermore, the number of patients with more than 2 motor nerves with CV slowing by the AAN or regression models ranges for demyelination and an additional criteria of more than one motor nerve with CV slowing in the demyelinating range with the corresponding F response in the demyelinating range by AAN criteria may be higher in patients of group A compared to group B (see, e.g., 70.3% versus 1.9%; p<0.0001 in FIG. 5A and FIG. 5B). The likelihood to have more than 2 motor nerves with CV slowing in the demyelinating range may be higher in the diabetic DSP group A compared to diabetic DSP group B (e.g., 0.84 versus 0.28, p<0.0001).

In some embodiments, the regression models and the correlation between CMAP amplitude as independent variable and CV as dependent variable for the two diabetic groups and the ALS group are shown in FIG. 5A and FIG. 5B.

In some embodiments, the Pearson correlation coefficients indicate correlation between the CMAP amplitude and CV in all studied groups as shown below in Table 8.

TABLE 8
Regression analysis of amplitude-dependent variation in distal latency, conduction
velocity and F response for Group A (diabetic DSP patients with at least one
motor nerve with CV slowing fulfilling the AAN criteria for acquired demyelination),
Group B (diabetic DSP patients with no motor nerves with CV slowing fulfilling
the AAN criteria for acquired demyelination) and ALS group.

| | | | | | |
|---|---|---|---|---|---|
| Change | Change | Change | Change | Change | Change |
| in DL | in CV | in F | in DL | in CV | in F |

Group A

| | | | | | | | |
|---|---|---|---|---|---|---|---|
| Median | 69 | 67 | 59 | Tibial | 48 | 48 | 39 |
| nerve (n) | | | | nerve (n) | | | |
| Intercept | 2.227 | 2.970 | 2.058 | Intercept | 90.655 | 6.768 | 2.049 |
| Slope | −0.095 | 3.801 | −0.001 | Slope | −0.037 | 1.336 | −0.002 |
| r^{a} | −0.249 | 0.439 | −0.016 | r^{a} | −0.136 | 0.518 | −0.097 |
| P-value | 0.0391 | 0.0002 | 0.9047 | P-value | 0.3560 | 0.0002 | 0.5580 |

Group B

| | | | | | | | |
|---|---|---|---|---|---|---|---|
| Median | 99 | 99 | 92 | Tibial | 86 | 86 | 80 |
| nerve (n) | | | | nerve (n) | | | |
| Intercept | 2.552 | 2.891 | 2.100 | Intercept | 93.132 | 8.400 | 2.041 |
| Slope | −0.256 | 0.129 | −0.006 | Slope | −0.078 | 0.899 | −0.005 |
| r^{a} | −0.445 | 0.269 | −0.305 | r^{a} | −0.443 | 0.398 | −0.332 |
| P-value | <0.0001 | 0.0071 | 0.0031 | P-value | <0.0001 | 0.0001 | 0.0026 |

ALS Group

| | | | | | | | |
|---|---|---|---|---|---|---|---|
| Median | 122 | 121 | 62 | Tibial | 135 | 104 | 88 |
| nerve (n) | | | | nerve (n) | | | |
| Intercept | 2.175 | 2.925 | 2.102 | Intercept | 97.573 | 8.926 | 2.020 |
| Slope | −0.133 | 0.141 | −0.008 | Slope | −0.071 | 0.767 | −0.004 |
| r^{a} | −0.601 | 0.494 | −0.357 | r^{a} | −0.352 | 0.369 | −0.362 |
| P-value | <0.0001 | <0.0001 | 0.004 | P-value | <0.0001 | 0.0001 | 0.005 |

DSP, Distal symmetrical polyneuropathy; ALS; Amyotrophic lateral sclerosis; CMAP, compound muscle action potential; CV, conduction velocity; DL, distal latency; F; F latency. CV, conduction velocity; DL, distal latency; n, number of motor nerves.
^{a}Pearson correlation coefficient.

In some embodiments, the slopes of the regression lines of CMAP amplitude (independent variable) versus CV (dependent variable) may be different from each other in the three studied groups (diabetic DSP group A, diabetic DSP group B and ALS group) in the ulnar nerve as shown in FIG. 6A and FIG. 6B. However, no difference may be observed in the slopes of the regression lines for the tibial, peroneal, and median nerves for the three groups. Furthermore, regression coefficients of Y-intercept (with CV as the dependent variable and CMAP amplitude as the independent variable) may be lower in the diabetic DSP group A compared to the diabetic DSP group B and to the ALS group in the tibial, peroneal and median nerves that cannot be explained by the difference in the slopes of these groups as shown in FIG. 6A and FIG. 6B and Table 8.

In some embodiments, there may be a CMAP independent CV slowing in intermediate motor nerve segments in diabetic DSP supporting a demyelination contribution in addition to large axonal loss. Furthermore, in some embodiments, the increase in the number of motor nerves with CV slowing in the AAN or regression analysis ranges in diabetic DSP patients with focal CV slowing fulfilling AAN demyelination criteria compared to diabetic DSP without focal demyelination and to axonal non-diabetic DSP is supportive of the presence of more diffuse demyelination process not enough severe to fulfill the AAN criteria for primary demyelination. The regression models characterize CV slowing in diabetic DSP and identify more diabetic DSP with CV slowing exceeding what is expected from axonal loss when conventional electrodiagnostic may be silent.

In some embodiments, the mean urine sPLA2 activity may be higher in diabetic subjects compared to subjects in a healthy control group (942.9±977.97 pmol/min/ml versus 591.6±390.15, p<0.05). Furthermore, there may be an increase in mean urine sPLA2 activity in diabetic DSP group A compared to group B (1328.3±1274.21 versus 673.8±576.93, p=0.0014). However, there may be no difference between sPLA2 activity in group B and the control group. Thus, there may be little or no correlation between urine sPLA2 activity and INCAT score.

In some embodiments, increased sPLA2 activity may be defined as an activity of, e.g., 1371.93 pmol/min/ml or other threshold value as detailed above, which may be equal to mean plus two standard deviations (Mean+2SD) of sPLA2 activity in 46 subjects in a control group.

In some embodiments, the number of patients with elevated sPLA2 activity having more than 2 motor nerves with CV slowing in the AAN or regression models ranges may be higher in patients of group A compared to group B (e.g., 35.1% versus 5.7%, p=0.0005). Furthermore, in this example study, 18.9% in diabetic DSP group A and no subjects in the diabetic DSP group B fulfilled an additional criterion of more than one motor nerve with CV slowing in the demyelinating range with the corresponding F response in the demyelinating range by AAN criteria.

In some embodiments, a combination of regression models with urine biomarker of neuroinflammation may better characterize demyelination in diabetic DSP and may eliminate overlaps between the diabetic DSP group with CV slowing presumed from a primary demyelination from diabetic axonal DSP.

In some embodiments, a Western blot analysis may be performed in patients' urine using medium and heavy filaments. The Western blot analysis of urine medium chain filament may be performed in the 70 subjects of this example study, e.g.: 22 subjects from a control group, 18 subjects from the diabetic group A and 30 subjects from the diabetic group B. The mean urine sPLA2 activity of the 22 subjects of the control group may be 626.0±378.39. The mean urine sPLA2 activity of the 18 diabetic subjects of group A may be 933.1±624.84. The mean urine sPLA2 activity of the 30 subjects of the diabetic group B may be 618.0±522.26. There may be a non-difference between the 2 diabetic groups (e.g., group A and group B), p=0.0665.

In some embodiments, in the diabetic group A, 27.8% of the subjects versus 10.0% of the subjects in diabetic group B had increased urine sPLA2 activity.

In some embodiments, the number of patients who undergo a urine neurofilaments testing and have more than 2 motor nerves with CV slowing in the demyelinating range either by AAN or regression equations criteria may be higher in the diabetic DSP group A compared to diabetic DSP group B (e.g., 83.3% versus 23.3%, p<0.0001).

In some embodiments, there may be a higher number of patients in the diabetic DSP group A who had positive urine heavy or medium chain neurofilaments compared to the diabetic DSP group B (e.g., 94.4% versus 60.0%, p<0.05). No subjects in the control group had both urine heavy and medium chain neurofilaments positive.

In some embodiments, in group A of this example, 77.8% of subjects may have positive urine heavy or medium chain neurofilaments and more than 2 motor nerves with CV in the demyelination range whereas only 10.0% of the subjects in group B of this example may have positive urine heavy or medium chain neurofilaments and more than 2 motor nerves with CV in the demyelination range, p<0.0001. Thus, in some embodiments, the presence of ongoing axonal loss in the diabetic DSP group A.

In some embodiments, the diabetic DSP group A of this example, 27.8% of the subjects and 3.3% of the subjects in the diabetic DSP group B of this example may have positive urine heavy or medium chain neurofilaments, more than 2 motor nerves with CV in the demyelination range and elevated urine sPLA2, p<0.05.

In some embodiments, diffuse motor nerve conduction slowing not fulfilling the AAN criteria for CIDP may be observed in diabetic DSP. In some embodiments, a better linear relationship between CMAP amplitude and CV is achieved with a combination of a square root transformation, fourth root transformation, or Log 10 transformation. This may be related to more variability in CV from which the regression models may be implemented. Conduction data may be derived from CIDP subjects, a primary acquired demyelinating disease with patchy demyelination across the motor nerve causing large variation of conduction not only between different motor nerves but in different segments of the same nerve.

In some embodiments, utilizing the regression models demonstrates the presence of higher number of subjects with 2 motor nerves with CV slowing in the demyelinating range in diabetic DSP subjects compared to axonal non-diabetic DSP subjects. The use of regression models may also be used to demonstrate that there is a higher number of subjects in this example in diabetic DSP group A (each subject in this group had at least one motor nerve with CV slowing by the AAN criteria) than diabetic DSP group B (no subject in this group had a motor nerve with CV slowing by AAN criteria) with more than 2 motor nerves with CV slowing in the demyelinating range. Furthermore, the likelihood to have more than 2 motor nerves with CV slowing in the demyelinating range is higher in the diabetic DSP group A compared to diabetic DSP group B. Therefore, in some embodiments, CV slowing may be beyond what is expected from an exclusive axonal loss, and the presence of one motor nerve with CV slowing in the demyelinating range by AAN criteria in diabetic DSP, may be indicative of generalized mild or moderate demyelinating process. In some embodiments, therefore, the regression models and/or the urinary testing may be used to detect demyelination even where the demyelination is not severe or extensive enough to affect proximal, intermediate, and distal peripheral nerve segments to fulfill the AAN criteria for demyelination.

In some embodiments, the presence of axonal loss in diabetic DSP group A and DSP group B may be supported by the presence of correlation between CMAP amplitude and CV in all studied motor nerves (see Table 8). However, the presence of an additional demyelination to axonal loss may be supported by the lower regression coefficients of Y-intercept and the difference in regression coefficients for the slopes of the regression lines of CMAP amplitude and CV between diabetic DSP group A and diabetic DSP group B as well as the ALS group. The latter two groups are presumed to have an exclusive axonal loss.

In some embodiments, compared to diabetic DSP group B, subjects in the DSP group A may have higher mean INCAT score and lower mean CMAP amplitude in all studied motor nerves. These demonstrates a more severe diabetic neuropathy in group A. Whether this is an evolving primary axonopathy with secondary demyelination or an evolving primary demyelination with secondary axonal loss, the combination of neurophysiological evidence of demyelination and axonal loss is associated with more severe diabetic neuropathy.

In some embodiments, because of their role in inflammation, sPLA2s are of interest as a biomarker of neuroinflammation in diabetic neuropathy. Phospholipase A2 (PLA2) hydrolyzes phosphatidylcholine to lysophosphatidylcholine and arachidonic acid. Lysophosphatidylcholine can induce myelin breakdown whereas arachidonic acid, via eicosanoids, can stimulate inflammatory responses. In some embodiments, immunohistochemical analysis of sPLA2 may demonstrate a role in Wallerian degeneration of peripheral nerves. sPLA2 activity is increased in type II diabetic patients with clinical cardiovascular disease compared to control groups and is associated with low-grade inflammation and endothelial activation. Furthermore, elevated glucose levels may enhance the expression of sPLA2 activity and proliferative responses in Schwann cells. Urine and plasma measurements reflect changes in systemic sPLA2 activity, depending on the timing of the measurement relative to the inflammatory stimulus. However, plasma measurements may be only reliable in a restricted window following such a stimulus because of the feedback mechanisms of sPLA2 products. Ongoing sPLA2 activity, that is specifically related to inflammation and neurodegeneration, can be more reliably measured in urine samples. For example, in relapsing/remitting multiple sclerosis (MS) subjects and in rodent models of MS, elevations in urinary sPLA2 measurements follow a predictable schedule relative to immunization and relapse symptoms.

In some embodiments, sPLA2 activity may be increased in clinical and experimental models of diabetes mellitus. In some embodiments, additionally, sPLA2 metabolites may be involved in neuroinflammation and in peripheral nerve Wallerian degeneration. Cyclooxygenase activity, a downstream component in PLA2 pathways, may be altered such that it is implicated in the pathogenesis of experimental diabetic neuropathy. In some embodiments, cyclooxygenase gene inactivation may have a protective effect against peripheral nerve dysfunction in experimental diabetes. Accordingly, urinary sPLA2 measurements have been used in the methods described herein.

Accordingly, as detailed herein, in some embodiments, mean urine sPLA2 activity is higher in diabetic patients compared to healthy subjects from control groups. Mean urine sPLA2 may be higher in the diabetic DSP group A for one or more of the above examples, the group with CV in the demyelinating range, compared to diabetic DSP group B, the group with no motor nerve with CV in the demyelinating range. Furthermore, when the regression models are combined with urine sPLA2 activity, 18.9% of subjects in the diabetic DSP group A and no subjects in the diabetic DSP group B had increased sPLA2 activity (sPLA2 activity more than 1371.93 pmol/min/ml) and had more than 2 motor nerves with CV slowing and at least one corresponding F in the demyelinating range.

In some embodiments, as detailed herein, the number of subjects with positive urine neurofilaments may be higher in the subgroup of diabetic DSP with elevated urine sPLA2 and CV in the demyelinating range indicating that neuroinflammation in this subgroup of diabetic DSP may be associated with active and ongoing axonal loss. Neurofilaments are major neuron specific cytoskeletal proteins responsible for maintaining cell structure and stability. Damage to peripheral nerve leads to systemic release of neurofilaments. Thus, in some embodiments, neurofilaments may be used as reliable biomarkers of axonal loss in inherited and acquired peripheral neuropathies.

In some embodiments, average INCAT scores may be higher in the subgroup of DSP with CV in the demyelinating range with elevated compared to the subgroup without CV in the demyelinating range indicative of a more severe neuropathy in the former group. The latter group had more evidence of a combination of axonal and demyelination on electrodiagnostic testing, elevated urine sPLA2 and active axonal loss as supported by the presence of urine neurofilaments. This emphasizes the need to identify and characterize the subgroup of diabetic DSP with positive markers of demyelination, neuroinflammation, and axonal loss that are responsive to therapeutic interventions.

In some embodiments, the use of regression models of CV slowing in diabetic DSP in combination with urine sPLA2 activity may more accurately and effectively identify a subgroup of diabetic DSP with contribution of acquired demyelination to peripheral nerve damage. The peripheral nerve damage in this subgroup may be active with an ongoing axonal loss. This is supported by the presence of urine neurofilaments as a marker of axonal loss. Accordingly, this subgroup may be determined as a candidate for immunotherapy or other therapeutic intervention that can prevent amputation as well as other complications of diabetic neuropathy for more effective treatment.

The aforementioned examples are, of course, illustrative, and not restrictive.

At least some aspects of the present disclosure will now be described with reference to the following numbered clauses.

1. A method for treating demyelinating neuropathy of a patient comprising:
placing a plurality of electrodes on a muscle area of the patient;
acquiring, via the plurality of electrodes, a compound muscle action potential (CMAP) measure of the muscle integrity in the muscle area of the patient;
obtaining a biological sample of the patient;
inputting a conduction measure associated with a CMAP amplitude comprised in the CMAP measure into a regression model to determine a conduction measure that is within a demyelinating range defined by the regression model;
determining, from the biological sample of the patient, a biomarker activity measure of the patient that is above a predetermined threshold;
determining that the patient has a demyelinating neuropathy based at least in part on:
the conduction measure that is within the demyelinating range defined by the regression model, and
the biomarker activity measure that is above the predetermined threshold; and
administering to the patient having the demyelinating neuropathy a therapeutically effective amount of at least one agent to treat the demyelinating neuropathy.

2. The method of clause 1, wherein the demyelinating neuropathy is a distal symmetric polyneuropathy.

3. The method of clause 1, wherein the demyelinating neuropathy is a peripheral neuropathy.

4. The method of clause 1, wherein the demyelinating neuropathy is a chronic inflammatory demyelinating polyneuropathy.

5. The method of clauses 1 to 3, wherein the biological sample is urine.

6. The method of clauses 1 to 5, wherein the biomarker activity measure is a neuroinflammatory biomarker.

7. The method of clauses 1 to 6, wherein the biomarker activity measure is an enzyme.

8. The method of clauses 1 to 7, wherein the biomarker activity measure is a secretory phospholipase 2 (sPLA2) enzyme.

9. The method of clauses 1 to 8, wherein the muscle area comprises a nerve selected from a tibial nerve, a peroneal nerve, a median nerve, an ulnar nerve, a radial nerve, and a sural nerve.

10. The method of clauses 1 to 9, wherein the plurality of electrodes comprises a surface electrode.

11. The method of clauses 1 to 10, wherein the plurality of electrodes comprises a needle-based electrode.

12. The method of clauses 1 to 11, wherein the needle-based electrode is selected from a group consisting of needle-based electrodes comprising a monopolar needle electrode, a concentric needle electrode, and a single-fiber needle electrode.

13. The method of clauses 5 to 12, wherein the demyelinating neuropathy is a chronic inflammatory demyelinating polyneuropathy and the at least one agent is an immune modulator agent.

14. The method of clauses 5 to 12, wherein the demyelinating neuropathy is a chronic inflammatory demyelinating polyneuropathy and the at least one agent is selected from a group consisting of immunoglobulin, glucocorticoid, and plasma.

15. The method of clauses 5 to 12, wherein the demyelinating neuropathy is a chronic inflammatory demyelinating polyneuropathy and the at least one agent is selected from a group consisting of azathioprine, cyclophosphamide, cyclosporine, etanercept, interferon alpha-2a, interferon beta-1a, mycophenolate mofetil, methotrexate, rituximab, and tacrolimus.

16. The method of clauses 5 to 12, wherein the demyelinating neuropathy is a distal symmetric polyneuropathy and the at least one agent is an antidepressant agent.

17. The method of clause 5 to 12, wherein the demyelinating neuropathy is a distal symmetric polyneuropathy and the at least one agent is an antiepileptic agent.

18. The method of clause 5 to 12, wherein the demyelinating neuropathy is a peripheral neuropathy and the at least one agent is an antidepressant agent.

19. The method of clauses 5 to 12, wherein the demyelinating neuropathy is a peripheral neuropathy and the at least one agent is an antiepileptic agent.

20. The method of claim 1, wherein the demyelinating neuropathy is a peripheral neuropathy and the at least one agent is an opioid-based agent.

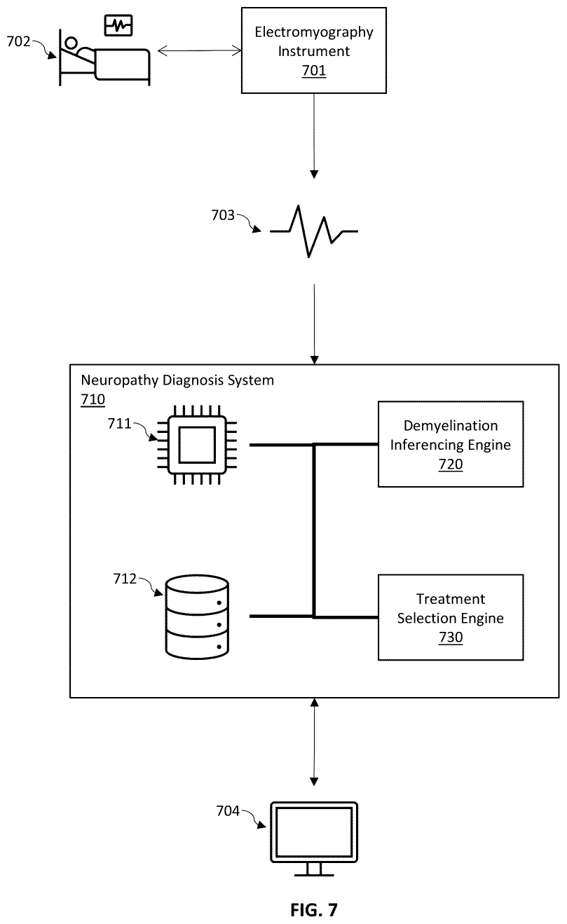

FIG. 7 is a block diagram of an exemplary computer-based system for demyelinating neuropathy detection and/or diagnosis in accordance with one or more embodiments of the present disclosure.

In some embodiments, as detailed above, an electromyography instrument 703, such as, e.g., electrodes may be used to gather electrodiagnostic signals from a patient 702. Electromyography is a technique for evaluating and recording the electrical activity produced by skeletal muscles. A clinician or other healthcare provider can place a set of electrodes on a muscle area of the patient 702. In some embodiments, the electrodes can be surface electrodes, for example, a single surface electrode, a pair of surface electrodes or an array of multiple surface electrodes. In some embodiments, the electrodes can be needle-based electrodes. In some embodiments, needle-based electrodes can be used to perform intramuscular electromyography. Similar to the surface electrodes, different number of needle-based electrodes can be placed on the patient, for example, one needle-based electrode, a pair of needle-based electrodes, or an array of multiple needle-based electrodes. Some examples of needle-based electrodes include monopolar needle electrodes, concentric needle electrodes, single-fiber needle electrode, and other suitable needle-based electrodes.

In some embodiments, the muscle area of the patient 702 can be any muscle area that can be used to capture electrical activity produced by skeletal muscles for example, the tibial nerve, the peroneal nerve, the median nerve, the ulnar nerve, the radial nerve, the sural nerve, or other suitable muscle areas.

In some embodiments, a compound muscle action potential (CMAP) 703 can be acquired via the set of electrodes placed on the muscle area of the patient 702. The CMAP 703 measure indicates a summation of a group of almost simultaneous action potentials from several muscle fibers located in the same muscle area of the patient 702. In some embodiments, latency, amplitude, duration, and area of the CMAP 703 measure can be calculated. An action potential occurs when the membrane potential of a specific cell location rapidly rises and falls: this depolarization then causes adjacent locations to similarly depolarize. Action potentials occur in certain patient 702 cells, called excitable cells, for example, neurons, muscle cells, endocrine cells, glomus cells, and other excitable cells.

In some embodiments, a biological sample can be obtained from the patient 702. For example, a urine sample can be obtained from the patient 702 to measure biomarkers activity contained in the urine sample. Examples of such biomarkers are secretory phospholipase 2 (sPLA2) enzymes or other suitable enzyme which control processes in skin and other organs, including inflammation and differentiation.

In some embodiments, CMAP 703 and/or additional patient data (e.g., the biomarkers of the biological sample, the latency, amplitude, duration, and area of the CMAP 703 measure, etc.) may be loaded into a neuropathy diagnosis system 710. The neuropathy diagnosis system 710 is configured to analyze the CMAP 703 and/or additional patient data to infer a neuropathy diagnosis based on a modelled demyelination, and recommend a treatment based on a selection of available treatments according to the diagnosis.

In some embodiments, the neuropathy and diagnosis system 710 may include hardware components such as a processor 711, which may include local or remote processing components. In some embodiments, the processor 711 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor 711 may include data-processing capacity provided by the microprocessor. In some embodiments, the microprocessor may include memory, processing, interface resources, controllers, and counters. In some embodiments, the microprocessor may also include one or more programs stored in memory.

Similarly, the neuropathy and diagnosis system 710 may include storage 712, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage 711 may include, e.g., a suitable non-transient computer readable medium such as, e.g., random access memory (RAM), read only memory (ROM), one or more buffers and/or caches, among other memory devices or any combination thereof.

In some embodiments, the neuropathy and diagnosis system 710 may implement computer engines for analyzing the CMAP 703 and/or additional patient data to infer a neuropathy diagnosis based on a modelled demyelination, and recommending a treatment based on a selection of available treatments according to the diagnosis. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, to analyze the CMAP 703 and/or additional patient data to infer a neuropathy diagnosis based on a modelled demyelination, the neuropathy diagnosis system 710 may include computer engines including, e.g., a demyelination inferencing engine 720. In some embodiments, the demyelination inferencing engine 720 may include dedicated and/or shared software components, hardware components, or a combination thereof. For example, the demyelination inferencing engine 720 may include a dedicated processor and storage. However, in some embodiments, the demyelination inferencing engine 720 may share hardware resources, including the processor 711 and storage 712 of the neuropathy and diagnosis system 710 via, e.g., a bus.

In some embodiments, the demyelination inferencing engine 720 may receive the CMAP 703 and/or additional patient data and model a demyelination present in the patient in order to determine neuropathy diagnosis. In some embodiments, a conduction measure, for example, conduction velocity associated with a CMAP amplitude can be calculated from the CMAP 703 measure. In some embodiments, a conduction measure such as the conduction velocity can be calculated as the difference of a proximal latency and distal latency and dividing the result by the distance between the proximal latency stimulating point and the distal latency stimulating point. In some embodiments, the demyelination inferencing engine 720 may instantiate a regression model to ingest the conduction measure and model the demyelination in the patient 702. In some embodiments, the conduction measure (e.g., conduction velocity or other suitable conduction measure as described above) can be inputted into the regression model to determine a conduction measure that is within a demyelinating range that is defined by the regression model as detailed above.

For example, in some embodiments, the regression model includes one or more regression functions with to link conduction velocity (CV) to distal CMAP amplitude of median, ulnar, peroneal, and tibial nerves. The normalized value for each attribute of motor nerve conduction may be expressed as a square root transformation, fourth root transformation, or Log 10 transformation to achieve a linear relationship correlating CMAP amplitude to CV. In some embodiments, the regression model may analyze the CMAP 703 according to the one or more functions to output a

the linear relationship between the CMAP amplitude of the corresponding CV and a resulting confidence interval, e.g., as detailed above. In some embodiments, the regression confidence intervals may be used in combination with AAN criteria for primary demyelination to capture conduction slowing not severe enough to fulfil the AAN demyelination criteria. The rate of CV slowing may be used to infer demyelination, and thus neuropathy, where the CV is outside of the confidence intervals because the presence of motor nerves with CV slowing by the AAN or regression model ranges may be indicative of CV slowing beyond what is expected from a pure axonal loss and could be related to a demyelinating process.

In some embodiments, as detailed above, the regression model may include, e.g., one or more predefined regression equations, customized/calibrated regression equations and/or one or more regression machine learning models trained on a corpus of training data. For example, for regression machine learning model(s), the regression machine learning model(s) ingests a feature vector that encodes features representative of CMAP measurements (e.g., CMAP amplitude, conduction velocity, or other CMAP measurement or any combination thereof). In some embodiments, the regression machine learning model(s) processes the feature vector with parameters to produces a prediction of a demyelination value range indicative of a degree of demyelination for CIDP, DSP and peripheral neuropathy, e.g., as measured by conduction slowing. In some embodiments, the parameters of the regression machine learning model(s) may be implemented in a suitable machine learning model including a regression machine learning model, such as, e.g., Linear Regression, Logistic Regression, Ridge Regression, Lasso Regression, Polynomial Regression, Bayesian Linear Regression (e.g., Naive Bayes regression), a recurrent neural network (RNN), decision trees, random forest, support vector machine (SVM), or any other suitable algorithm for predicting output values based on input values.

In some embodiments, the regression machine learning model(s) processes the features encoded in the feature vector by applying the parameters of the regression machine learning model to produce a model output vector. In some embodiments, the model output vector may be decoded to generate one or more numerical output values indicative of the range of demyelination. In some embodiments, the model output vector may include or may be decoded to reveal the output value(s) based on a modelled correlation between the feature vector and a target output. In some embodiments, the numerical output may represent the conduction slowing and/or a range of values of the conduction slowing.

In some embodiments, the parameters of the regression machine learning model(s) may be trained based on known outputs. For example, the CMAP measurements may be paired with a target value or known value to form a training pair, such as a historical CMAP measurement of a patient in a study and an observed result and/or human annotated value representing a data point in the relationship between the historical CMAP measurement and conduction slowing. In some embodiments, the CMAP measurement may be provided to the regression machine learning model(s), e.g., encoded in a feature vector, to produce a predicted output value. In some embodiments, an optimizer associated with the regression machine learning model(s) may then compare the predicted output value with the known output of a training pair including the historical CMAP measurement to determine an error of the predicted output value. In some embodiments, the optimizer may employ a loss function, such as, e.g., Hinge Loss, Multi-class SVM Loss, Cross Entropy Loss, Negative Log Likelihood, or other suitable classification loss function to determine the error of the predicted output value based on the known output.

In some embodiments, upon determining a degree of demyelination and inferring a neuropathy diagnosis based thereon, the neuropathy diagnosis system 710 may use the treatment selection engine 730 to recommend a therapeutically effective treatment based on the demyelination, the diagnosis and/or a library of potential treatment options, e.g., stored in the storage 712. In some embodiments, library of potential treatment options may be indexed by type of neuropathy, underlying condition, degree of demyelination, severity of neuropathy, CV and/or CV slowing, among other data associated with the neuropathy diagnosis.

In some embodiments, to recommending a treatment based on a selection of available treatments according to the diagnosis, the neuropathy diagnosis system 710 may include computer engines including, e.g., a treatment selection engine 730. In some embodiments, the treatment selection engine 730 may include dedicated and/or shared software components, hardware components, or a combination thereof. For example, the treatment selection engine 730 may include a dedicated processor and storage. However, in some embodiments, the treatment selection engine 730 may share hardware resources, including the processor 711 and storage 712 of the neuropathy and diagnosis system 710 via, e.g., a bus.

In some embodiments, the neuropathy diagnosis system 710 may output the treatment recommendation to a user computing device 704 to recommend to a user (e.g., doctor, nurse, practitioner, or other caregiver associated with the patient 702), the therapeutically effective treatment. Such output may cause the user computing device 704 to render a graphical user interface that presents the recommendation to the user. Accordingly, the user may then administer a therapeutically effective treatment to the patient 702 based, at least in part, on the recommendation provided by the neuropathy diagnosis system 710.

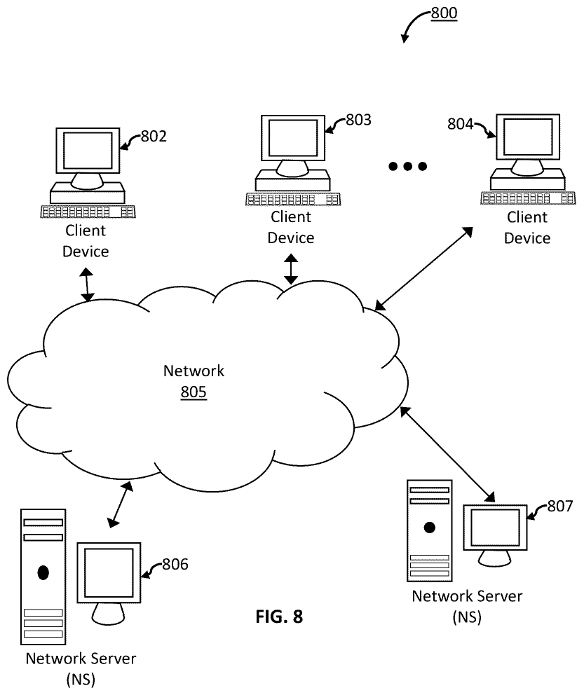

FIG. 8 depicts a block diagram of an exemplary computer-based system and platform 800 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 800 may be configured to manage a large number of members and concurrent transactions, as detailed herein. In some embodiments, the exemplary computer-based system and platform 800 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 8, client device 802, client device 803 through client device 804 (e.g., clients) of the exemplary computer-based system and platform 800 may include virtually any computing device capable of receiving and sending a message over a network (e.g., cloud network), such as network 805, to and from another computing device, such as servers 806 and 807, each other, and the like. In some embodiments, the client devices 802 through 804 may be personal computers, multiprocessor systems, microprocessor-based or programmable consumer electronics, network PCs, and the like. In some embodiments, one or more client devices within client devices 802 through 804 may include computing devices that typically connect using a wireless communications medium such as cell phones, smart phones, pagers, walkie talkies, radio frequency (RF) devices, infrared (IR) devices, CBs citizens band radio, integrated devices combining one or more of the preceding devices, or virtually any mobile computing device, and the like. In some embodiments, one or more client devices within client devices 802 through 804 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, OFDM, OFDMA, LTE, satellite, ZigBee, etc.). In some embodiments, one or more client devices within client devices 802 through 804 may include may run one or more applications, such as Internet browsers, mobile applications, voice calls, video games, videoconferencing, and email, among others. In some embodiments, one or more client devices within client devices 802 through 804 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a client device within client devices 802 through 804 may be specifically programmed by either Java, .Net, QT, C, C++, Python, PHP and/or other suitable programming language. In some embodiment of the device software, device control may be distributed between multiple standalone applications. In some embodiments, software components/applications can be updated and redeployed remotely as individual units or as a full software suite. In some embodiments, a client device may periodically report status or send alerts over text or email. In some embodiments, a client device may contain a data recorder which is remotely downloadable by the user using network protocols such as FTP, SSH, or other file transfer mechanisms. In some embodiments, a client device may provide several levels of user interface, for example, advance user, standard user. In some embodiments, one or more client devices within client devices 802 through 804 may be specifically programmed include or execute an application to perform a variety of possible tasks, such as, without limitation, messaging functionality, browsing, searching, playing, streaming, or displaying various forms of content, including locally stored or uploaded messages, images and/or video, and/or games.

In some embodiments, the exemplary network 805 may provide network access, data transport and/or other services to any computing device coupled to it. In some embodiments, the exemplary network 805 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 805 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 805 may include and implement, as an alternative or in conjunction with one or more of the above, a WiMAX architecture defined by the WiMAX forum. In some embodiments and, optionally, in combination of any embodiment described above or below, the exemplary network 805 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 805 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, OFDM, OFDMA, LTE, satellite and any combination thereof. In some embodiments, the exemplary network 805 may also include mass storage, such as network attached storage (NAS), a storage area network (SAN), a content delivery network (CDN) or other forms of computer or machine readable media.

In some embodiments, the exemplary server 806 or the exemplary server 807 may be a web server (or a series of servers) running a network operating system, examples of which may include but are not limited to Apache on Linux or Microsoft IIS (Internet Information Services). In some embodiments, the exemplary server 806 or the exemplary server 807 may be used for and/or provide cloud and/or network computing. Although not shown in FIG. 8, in some embodiments, the exemplary server 806 or the exemplary server 807 may have connections to external systems like email, SMS messaging, text messaging, ad content providers, etc. Any of the features of the exemplary server 806 may be also implemented in the exemplary server 807 and vice versa.

In some embodiments, one or more of the exemplary servers 806 and 807 may be specifically programmed to perform, in non-limiting example, as authentication servers, search servers, email servers, social networking services servers, Short Message Service (SMS) servers, Instant Messaging (IM) servers, Multimedia Messaging Service (MMS) servers, exchange servers, photo-sharing services servers, advertisement providing servers, financial/banking-related services servers, travel services servers, or any similarly suitable service-base servers for users of the client devices 801 through 804.

In some embodiments and, optionally, in combination of any embodiment described above or below, for example, one or more exemplary computing client devices 802 through 804, the exemplary server 806, and/or the exemplary server 807 may include a specifically programmed software module that may be configured to send, process, and receive information using a scripting language, a remote procedure call, an email, a tweet, Short Message Service (SMS), Multimedia Message Service (MMS), instant messaging (IM), an application programming interface, Simple Object Access Protocol (SOAP) methods, Common Object Request Broker Architecture (CORBA), HTTP (Hypertext Transfer Protocol), REST (Representational State Transfer), SOAP (Simple Object Transfer Protocol), MLLP (Minimum Lower Layer Protocol), or any combination thereof.

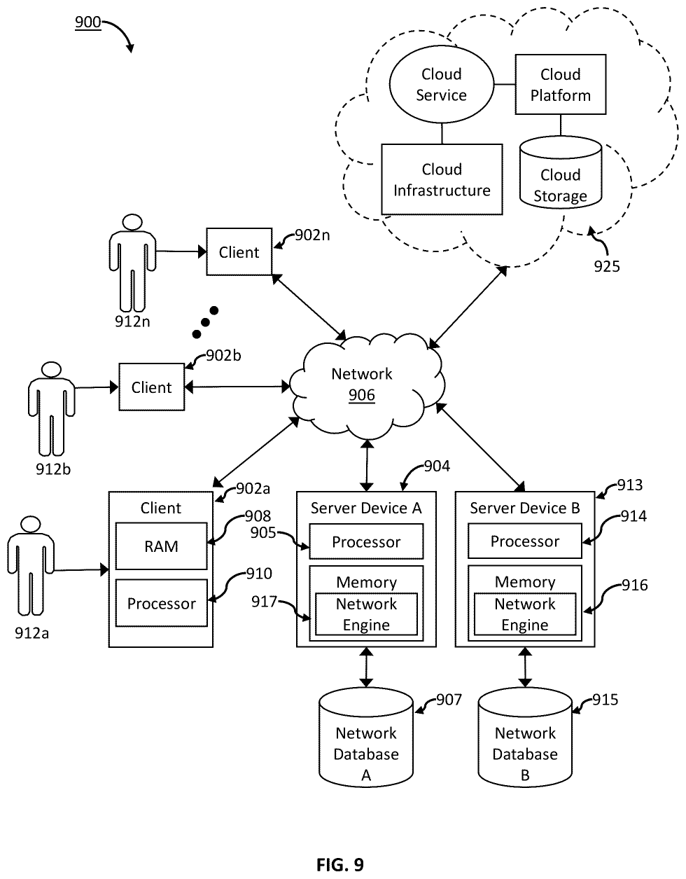

FIG. 9 depicts a block diagram of another exemplary computer-based system and platform 900 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the client device 902a, client device 902b through client device 902n shown each at least includes a computer-readable medium, such as a random-access memory (RAM) 908 coupled to a processor 910 or FLASH memory. In some embodiments, the processor 910 may execute computer-executable program instructions stored in memory 908. In some embodiments, the processor 910 may include a microprocessor, an ASIC, and/or a state machine. In some embodiments, the processor 910 may include, or may be in communication with, media, for example computer-readable media, which stores instructions that, when executed by the processor 910, may cause the processor 910 to perform one or more steps described herein. In some embodiments, examples of computer-readable media may include, but are not limited to, an electronic, optical, magnetic, or other storage or transmission device capable of providing a processor, such as the processor 910 of client device 902a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape, or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, and etc.

In some embodiments, client devices 902a through 902n may also comprise a number of external or internal devices such as a mouse, a CD-ROM, DVD, a physical or virtual keyboard, a display, or other input or output devices. In some embodiments, examples of client devices 902a through 902n (e.g., clients) may be any type of processor-based platforms that are connected to a network 906 such as, without limitation, personal computers, digital assistants, personal digital assistants, smart phones, pagers, digital tablets, laptop computers, Internet appliances, and other processor-based devices. In some embodiments, client devices 902a through 902n may be specifically programmed with one or more application programs in accordance with one or more principles/methodologies detailed herein. In some embodiments, client devices 902a through 902n may operate on any operating system capable of supporting a browser or browser-enabled application, such as Microsoft™, Windows™, and/or Linux. In some embodiments, client devices 902a through 902n shown may include, for example, personal computers executing a browser application program such as Microsoft Corporation's Internet Explorer™, Apple Computer, Inc.'s Safari™, Mozilla Firefox, and/or Opera. In some embodiments, through the member computing client devices 902a through 902n, user 912a, user 912b through user 912n, may communicate over the exemplary network 906 with each other and/or with other systems and/or devices coupled to the network 906. As shown in FIG. 9, exemplary server devices 904 and 913 may include processor 905 and processor 914, respectively, as well as memory 917 and memory 916, respectively. In some embodiments, the server devices 904 and 913 may be also coupled to the network 906. In some embodiments, one or more client devices 902a through 902n may be mobile clients.

In some embodiments, at least one database of exemplary databases 907 and 915 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

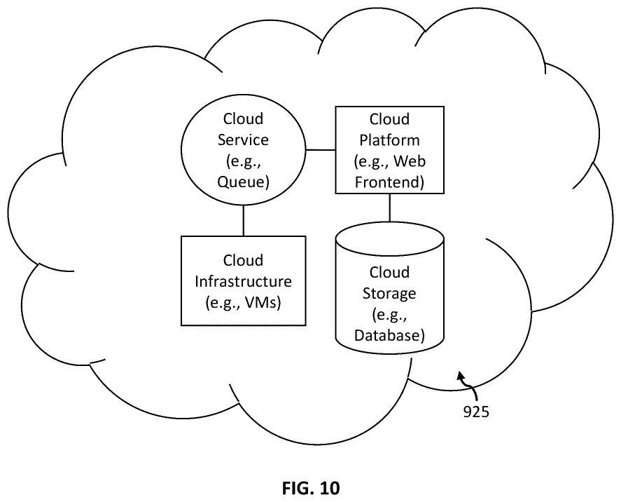

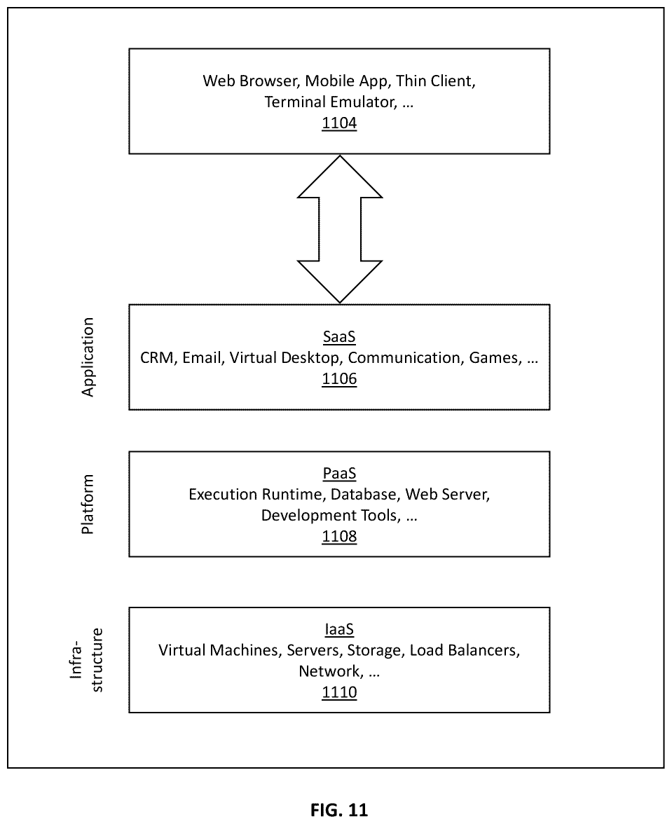

In some embodiments, the exemplary inventive computer-based systems/platforms, the exemplary inventive computer-based devices, and/or the exemplary inventive computer-based components of the present disclosure may be specifically configured to operate in a cloud computing/architecture 925 such as, but not limiting to: infrastructure a service (IaaS) 1110, platform as a service (PaaS) 1108, and/or software as a service (SaaS) 1106 using a web browser, mobile app, thin client, terminal emulator or other endpoint 1104. FIGS. 10 and 11 illustrate schematics of exemplary implementations of the cloud computing/architecture(s) in which the exemplary inventive computer-based systems/platforms, the exemplary inventive computer-based devices, and/or the exemplary inventive computer-based components of the present disclosure may be specifically configured to operate.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical, or other forms of propagated signals (e.g., carrier waves, infrared signals, digital signals, etc.), and others.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” may be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

As used herein, the term “user” shall have a meaning of at least one user. In some embodiments, the terms “user”, “patient,” “subscriber” “consumer” or “customer” may be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

The aforementioned examples are, of course, illustrative, and not restrictive.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiment of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

1. A method for treating demyelinating neuropathy of a patient comprising:
placing a plurality of electrodes on a muscle area of the patient;
acquiring, via the plurality of electrodes, a compound muscle action potential (CMAP) measure of muscle integrity in the muscle area of the patient;
obtaining a biological sample of the patient;
inputting a conduction measure associated with a CMAP amplitude comprised in the CMAP measure into a regression model to determine a conduction measure that is within a demyelinating range defined by the regression model;
determining, from the biological sample of the patient, a biomarker activity measure of the patient that is above a predetermined threshold;
determining that the patient has a demyelinating neuropathy based at least in part on:
a) the conduction measure that is within the demyelinating range defined by the regression model, and
b) the biomarker activity measure that is above the predetermined threshold; and
administering to the patient having the demyelinating neuropathy a therapeutically effective amount of at least one agent to treat the demyelinating neuropathy.

2. The method of claim 1, wherein the demyelinating neuropathy is a distal symmetric polyneuropathy.

3. The method of claim 1, wherein the demyelinating neuropathy is a peripheral neuropathy.

4. The method of claim 1, wherein the demyelinating neuropathy is a chronic inflammatory demyelinating polyneuropathy.

5. The method of claim 1, wherein the biological sample is urine.

6. The method of claim 1, wherein the biomarker activity measure is a neuroinflammatory biomarker.

7. The method of claim 1, wherein the biomarker activity measure is an enzyme.

8. The method of claim 1, wherein the biomarker activity measure is a secretory phospholipase 2 (sPLA2) enzyme.

9. The method of claim 1, wherein the muscle area comprises a nerve selected from a tibial nerve, a peroneal nerve, a median nerve, an ulnar nerve, a radial nerve, and a sural nerve.

10. The method of claim 1, wherein the plurality of electrodes comprises a surface electrode.

11. The method of claim 1, wherein the plurality of electrodes comprises a needle-based electrode.

12. The method of claim 11, wherein the needle-based electrode is selected from a group consisting of needle-based electrodes comprising a monopolar needle electrode, a concentric needle electrode, and a single-fiber needle electrode.

13. The method of claim 1, wherein the demyelinating neuropathy is a chronic inflammatory demyelinating polyneuropathy and the at least one agent is an immune modulator agent.

14. The method of claim 1, wherein the demyelinating neuropathy is a chronic inflammatory demyelinating polyneuropathy and the at least one agent is selected from a group consisting of immunoglobulin, glucocorticoid, and plasma.

15. The method of claim 1, wherein the demyelinating neuropathy is a chronic inflammatory demyelinating polyneuropathy and the at least one agent is selected from a group consisting of azathioprine, cyclophosphamide, cyclosporine, etanercept, interferon alpha-2a, interferon beta-1a, mycophenolate mofetil, methotrexate, rituximab, and tacrolimus.

16. The method of claim 1, wherein the demyelinating neuropathy is a distal symmetric polyneuropathy and the at least one agent is an antidepressant agent.

17. The method of claim 1, wherein the demyelinating neuropathy is a distal symmetric polyneuropathy and the at least one agent is an antiepileptic agent.

18. The method of claim 1, wherein the demyelinating neuropathy is a peripheral neuropathy and the at least one agent is an antidepressant agent.

19. The method of claim 1, wherein the demyelinating neuropathy is a peripheral neuropathy and the at least one agent is an antiepileptic agent.

20. The method of claim 1, wherein the demyelinating neuropathy is a peripheral neuropathy and the at least one agent is an opioid-based agent.
